(19) United States
(12) Patent Application Publication
(10) Pub. No.: US 2026/0062684 A1
(43) Pub. Date: Mar. 5, 2026

(54) In Vivo Genetic Engineering of Antigen Responsive Cells

(71) Applicant: THOMAS JEFFERSON UNIVERSITY, Philadelphia, PA (US)

(72) Inventor: Vitali Alexeev, Philadelphia, PA (US)

(21) Appl. No.: 19/093,848

(22) Filed: Mar. 28, 2025

Related U.S. Application Data
(63) Continuation of application No. 17/271,314, now abandoned.
(60) Provisional application No. 62/725,433, filed on Aug. 31, 2018.

(51) Int. Cl. C12N 9/12; A61K 40/11; A61K 40/31; A61K 40/32; A61K 40/42; C07K 14/52; C12N 15/87

(52) U.S. Cl. CPC C12N 9/12; A61K 40/11; A61K 40/31; A61K 40/32; A61K 40/4211; A61K 40/4245; A61K 40/4261; C07K 14/52; C12N 15/87; A61K 2239/31; A61K 2239/38; A61K 2239/48; A61K 2239/57; C12N 2510/00; C12Y 207/00

(57) ABSTRACT

The invention provides methods for genetically engineering T cells in vivo comprising administering to the subject a cytokine or nucleic acid molecule encoding a cytokine to recruit the subject's T cells to the administration site; followed by administration of a nucleic acid molecule encoding an antigen receptor. In some instances, the method also includes administering an integrase or nucleic acid molecule encoding an integrase to integrate the sequence encoding the antigen receptor into the DNA of the recruited T cells.

### CROSS REFERENCE TO RELATED APPLICATIONS

This application claims priority to U.S. Provisional Application No. 62/725,433, filed Aug. 31, 2018 which is hereby incorporated by reference herein in its entirety.

### BACKGROUND OF THE INVENTION

Cancer immunotherapy via adoptive cell transfer (ACT) of lymphocytes genetically engineered to express tumor-specific T cell receptor (TCR) or chimeric antigen receptor (CAR) involves multiple steps including (i) isolation of patient-derived T cells from peripheral blood (PBL, peripheral blood lymphocytes) of cancer patient, (ii) propagation of these cells ex vivo, (III) infection of the cells with retro (lenti) virus encoding TCR or CAR, (iv) propagation of infected cells ex vivo, (v) ACT of the resultant T cells into pre-conditioned patient.

All these procedures are labor-retaining, time-consuming and expensive. Prior to ACT, patients are undergoing lympho-depleting nonmyeloablative chemotherapy. ACT is done by the infusion of a large number of cells (1×10¹⁰ cells per treatment). Infusion such large quantities often results in side-effects including so-called “cytokine storm” cause, possibly, by cytokines released by infused activated T cells.

ACT using lymphocytes genetically engineered to express tumor-specific TCR or CAR demonstrated high (50% to 90%) remission rates in patients with advanced (stage IV) cancers. To date, several recombinant melanoma-specific TCR, including tyrosinase-specific TCR (Tyr-TCR) (Frankel et al., J Immunol 2010, 184:5988-5998) have been cloned, optimized and used in more than 20 clinical studies in the United States alone to eradicate bulky malignant lesions of advanced melanoma (Park et al, Trends in biotechnology 2011, 29:550-557; Phan et al., Cancer control: journal of the Moffitt Cancer Center 2013, 20:289-297; Frankel et al., J Immunol 2010, 184:5988-5998; Perro et al., Gene therapy 2010, 17:721-732)). Several CAR molecules designed to target solid and liquid tumors were also established and tested in preclinical and clinical settings (Jennrich et al., Journal of virology 2012, 86:3436-3445. 3302526; Beard et al., Journal for immunotherapy of cancer 2014, 2:25. 4155770). Recently, ACT with CAR-T cells recognizing common B cell antigen, CD19, were approved by the FDA for the treatment of B-cell precursor acute lymphoblastic leukemia (First-Ever CAR T-cell Therapy Approved in U.S, Cancer discovery 2017, 7:OF1). Despite rapid progress in the cloning, design and optimization of novel tumor-targeting receptors, several drawbacks, including inability to rapidly alter treatment regimen, off- and on-target toxicities, labor intensive and time-consuming production of the recombinant T cells ex vivo and high cost of the treatment hamper the advancement of the recombinant T cell-based therapies to general practice. However, several drawbacks including inability to rapidly alter treatment regimen, off- and on-target toxicities, and high costs of the recombinant T cell production hamper the advancement of this modality to general practice.

Thus, there is a need in the art for improved compositions and methods to utilize recombinant T-cell based therapies for the treatment and prevention of cancer. The present invention satisfies this unmet need.

### SUMMARY OF THE INVENTION

In one aspect, the present invention provides a method for treating cancer in a subject in need thereof. In one embodiment, the method comprises administering a cytokine composition at a treatment site of the subject thereby recruiting at least one T cell to the treatment site; and administering an antigen receptor composition to the subject to genetically modify the recruited at least one T cell to express an antigen receptor.

In one embodiment, the cytokine composition comprises a recombinant cytokine or nucleic acid molecule encoding a cytokine. In one embodiment, the cytokine is selected from the group consisting of CCL2, CCL3, CCL4, CCL5, macrophage inflammatory proteins (MIP-1α), CXCL9, CXCL10, CXCL12, CXCL16, CCL17, CCL19, CCL20, CCL21, CCL22, and CCL27.

In one embodiment, the antigen receptor composition comprises an isolated nucleic acid molecule comprising a nucleic acid sequence encoding an antigen receptor. In one embodiment, the antigen receptor is a T cell receptor (TCR) or chimeric antigen receptor (CAR). In one embodiment, the antigen receptor composition is administered at the treatment site.

In one embodiment, the method further comprises administering an integration composition to the subject, wherein the integration composition induces the integration of the nucleic acid sequence encoding the antigen receptor into the DNA of the recruited at least one T cell. In one embodiment, the integration composition comprises an integrase, nucleic acid molecule encoding an integrase, recombinase, or nucleic acid molecule encoding a recombinase.

In one embodiment, administration of the cytokine composition recruits a diverse population of T cells. In one embodiment, administration of the cytokine composition recruits a pre-defined subset of T cells.

In one embodiment, the treatment site is the skin or a tumor of the subject.

In one embodiment, the nucleic acid molecule encoding a cytokine is administered using electroporation. In one embodiment, the nucleic acid molecule encoding an antigen receptor is administered using electroporation. In one embodiment, the nucleic acid molecule encoding an integrase or the nucleic acid molecule encoding a recombinase is administered using electroporation.

In one aspect, the present invention provides a method for generating a tumor-specific T cell in a subject. In one embodiment, the method comprises administering a cytokine composition at a treatment site of the subject thereby recruiting at least one T cell to the treatment site; and administering an antigen receptor composition to the subject to genetically modify the recruited at least one T cell to express an antigen receptor that binds to a tumor-specific antigen.

In one embodiment, the cytokine composition comprises a recombinant cytokine or nucleic acid molecule encoding a cytokine. In one embodiment, the cytokine is selected from the group consisting of CCL2, CCL3, CCL4, CCL5, macrophage inflammatory proteins (MIP-1α), CXCL9, CXCL10, CXCL12, CXCL16, CCL17, CCL19, CCL20, CCL21, CCL22, and CCL27.

In one embodiment, the antigen receptor composition comprises an isolated nucleic acid molecule comprising a nucleic acid sequence encoding an antigen receptor. In one embodiment, the antigen receptor is a T cell receptor (TCR) or chimeric antigen receptor (CAR). In one embodiment, the antigen receptor composition is administered at the treatment site.

In one embodiment, the method further comprises administering an integration composition to the subject, wherein the integration composition induces the integration of the nucleic acid sequence encoding the antigen receptor into the DNA of the recruited at least one T cell. In one embodiment, the integration composition comprises an integrase, nucleic acid molecule encoding an integrase, recombinase, or nucleic acid molecule encoding a recombinase.

In one embodiment, administration of the cytokine composition recruits a diverse population of T cells. In one embodiment, administration of the cytokine composition recruits a pre-defined subset of T cells.

In one embodiment, the treatment site is the skin or a tumor of the subject.

In one embodiment, the nucleic acid molecule encoding a cytokine is administered using electroporation. In one embodiment, the nucleic acid molecule encoding an antigen receptor is administered using electroporation. In one embodiment, the nucleic acid molecule encoding an integrase or the nucleic acid molecule encoding a recombinase is administered using electroporation.

### BRIEF DESCRIPTION OF THE DRAWINGS

The following detailed description of embodiments of the invention will be better understood when read in conjunction with the appended drawings. It should be understood that the invention is not limited to the precise arrangements and instrumentalities of the embodiments shown in the drawings.

FIG. 1 depicts a schematic of an exemplary method of the present invention. T cell genetic engineering in vivo eliminates ex vivo manipulation with patient-derived T cells making this approach generic and cost-effective. It permits multiple treatment, rapid alteration to treatment regimen, treatment of the tumors with different tumor-targeting T cells. It eliminates side-effects associated with the infusion of large quantities of the activated T cells. DNA electroporation procedure takes a few seconds. Multiple sites could be treated at the same time.

FIG. 2 depicts the prior art strategy for T cell genetic engineering ex vivo. The procedure utilizes GMP facilities for tissue culture, use of viruses encoding T cell receptors, necessity of lymphodepletion.

FIG. 3A and FIG. 3B depict two plasmids encoding tyrosinase-specific T cell receptor (TCR) with attB integration sites. (FIG. 3A)-map of a plasmid comprise of DNA sequences encoding a and B chains to tyrosinase-specific TCR translationally linked via P2a ribozyme skipping element and a full-length attB site for psiC31 integrase-mediated insertion of the plasmid into mammalian (human) genome. (FIG. 3B)-map of the Tyr-TCR plasmid with addition of 4-1BB an CD3 zeta signaling domains, which enhance T cell activity.

FIG. 4A-FIG. 4F depict analysis of ΦC31-integrase-mediated gene transfer, Tyr-TCR expression and functional activity. (FIG. 4A and FIG. 4B) GFP expression in CD4+ and CD8+ T cells 24 h after nucleofection; (FIG. 4C and FIG. 4D) GFP expression in primary T cells 14 days after nucleofection assessed by FACS and direct fluorescence (FIG. 4D). Shaded profiles in (FIG. 4A-FIG. 4C)-control T cells; green profiles-GFP+ cells. (FIG. 4E) CTL activity of the Tyr-TCR-transduced T cells against HLA-A2+ Tyrosinase+ melanoma. (FIG. 4F) Tyrosinase-tetramer binding to CD8+ T cells transduced with Tyr-TCR expression vector one week after ΦC31-mediated gene transfer.

FIG. 5A-FIG. 5D depicts intradermal (ID) and intratumoral (IT) localization of CD3+ T cells after in vivo electroporation of a plasmid DNA encoding secondary lymphoid chemokine, CCL21, and in vivo transfer of the Tyr-TCR transgene. (FIG. 5A) Representative micrographs depicting indirect immunofluorescent detection of CD3+ T cells in the skin and the tumors in control and CCL21 pre-conditioned (treated) regions. (FIG. 5B) Quantitation of CD3+ cells on images showing statistically significant difference (p<0.05) between control and CCL21-treated skin and tumors. (FIG. 5C) In vivo live animal imaging showing expression of the Tyr-TCR-DsRed reporter plasmid electroporated into CCL21 pre-conditioning skin. Expression of the transgene is detected by expression of the red fluorescent reporter (DsRed), which is translationally linked to the Tyr-TCR-encoding DNA. Micrograph to the right showed DsRed expression in T cells recruited to the skin. (FIG. 5D) Quantitation of the intradermal cells showing that about 50% of CD3+ T cells recovered from the treated skin express DsRed reporter.

FIG. 6A-FIG. 6B depicts the results of experiments investigating chemokine-mediated T cells recruitment and gene transfer. (FIG. 6A) Indirect immunofluorescent detection of the CD3+ T cells (green) in chemokine-treated skin and melanoma lesions (as indicated). Chemokines are shown above the panels. Blue-DAPI nuclear staining. (FIG. 6B) Quantitation of T cell infiltrates: C-Control, 1-CCL22, 2-CCL5, 3-CCL2, 4-CCL21, 5-CCL21/22. Statistical significance is shown as p<0.05.

FIG. 7A-FIG. 7C depict recombinant TCR expression and activity in T cells recovered from the treated skin. (FIG. 7A and FIG. 7B) FACS-based profiles and density plots showing equal distribution of the CD4+ and CD8+ T cells recovered from the skin (FIG. 7A) and expression of the recombinant tyrosinase-specific TCR (Tyr-TCR) in approximately 50% of CD4+ and CD8+ T cells 48 h after Tyr-TCR gene transfer (FIG. 7B) targeting of the establish B16/A2 melanoma by intratumoral T cell genetic engineering. (FIG. 7B) Analysis of cytotoxicity of the recovered cells against HLA-A2-positive (B16/A2) and negative (B16F0) targets, as indicated.

FIG. 8A-FIG. 8C depicts analysis of tumor growth in two cohorts of control and experimental mice. Graphs show: (FIG. 8A) average tumor volumes, and volumes of individual tumors growing in mock-treated (FIG. 8B) and TCR-treated (FIG. 8C) lesions.

FIG. 9A-FIG. 9C depicts the results of immunotargeting of the established B16/A2 melanoma using intratumoral T cell genetic engineering. (FIG. 9A and FIG. 9B) Local depigmentation at site of tumor treatment (FIG. 9A) and challenging inoculation of the tumor cells (FIG. 9B). Magnified view shown on inserts. (FIG. 9C) Kaplan-Meier survival analysis in control and Tyr-TCR-treated mice.

FIG. 10A-FIG. 10C depicts recombinant TCR design and the results of experiments investigating recombinant TCR activity in vitro. FIG. 10A depicts original and two modified and tested constructs as indicated. FIG. 10B and FIG. 10C illustrates the results of ELISA analysis of IFNγ and IL-2 secretion from Tyr-TCR transduced T cells. TCR constructs and target cells indicated below the columns. Data is presented as a mean of 3 independent experiments±SD.

FIG. 11A-FIG. 11D depicts the results of experiments demonstrating in vitro analysis of the CSPG-4 CAR-T cells activity. (FIG. 11A) Diagram depicting current CSPG-4 CAR construct; (FIG. 11B) FACS-based quantitation of the cell surface antigens in 4 different melanoma cell line (as indicated above the panels). Estimated number of antigens per cells (in thousands) is shown inside the panels; (FIG. 11C) CTL activity of the in vitro engineered CSPG-4-CAR T cells against selected melanoma cells; (FIG. 11D) degranulation assay against selected melanoma cells. Data on (FIG. 11C and FIG. 11D) is presented as a mean±SD.

FIG. 12 depicts the results of experiments investigating CTL activity of the in vitro engineered CD19-CAR T cells against CD19+ and CD19−(control) targets (as indicated).

### DETAILED DESCRIPTION

The invention is based in part on the development of compositions and methods for generating immunoresponsive cells in vivo. In one embodiment, the method comprises locally introducing to a subject a first composition comprising one or more cytokines, or one or more nucleic acid molecules encoding one or more cytokines, to recruit one or more naïve immunoresponsive cells to the administration site. In one embodiment, the method further comprises, subsequent administration of one or more compositions comprising a nucleic acid molecule encoding a recombinant T cell receptor (TCR) or chimeric antigen receptor (CAR). In one embodiment, the method comprises administration of one or more recombinase or integrase, or nucleic acid molecule encoding a recombinase or integrase. In one embodiment, the methods of the invention generate an active immunoresponsive cell in vivo, where the immunoresponsive cell is modified to express a desired antigen receptor that binds an antigen. Therefore, in one embodiment, the invention relates to methods for in vivo immunotherapy.

### Definitions

Unless defined otherwise, all technical and scientific terms used herein have the same meaning as commonly understood by one of ordinary skill in the art to which this invention belongs.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

As used herein, the term “Chimeric Antigen Receptor” or alternatively a “CAR” refers to a recombinant polypeptide construct comprising at least an extracellular antigen binding domain, a transmembrane domain and a cytoplasmic signaling domain comprising a functional signaling domain derived from a stimulatory molecule as defined herein. In one aspect, the stimulatory molecule is the zeta chain associated with the T cell receptor complex. In one aspect, the intracellular signaling domain further comprises one or more functional signaling domains derived from at least one costimulatory molecule as defined below. In one aspect, the costimulatory molecule is chosen from 4-1BB (i.e., CD137) and/or CD28. In one aspect, the CAR comprises a chimeric fusion protein comprising an extracellular antigen recognition domain, a transmembrane domain and a cytoplasmic signaling domain comprising a functional signaling domain derived from a stimulatory molecule. In one aspect, the CAR comprises a chimeric fusion protein comprising an extracellular antigen recognition domain, a transmembrane domain and a cytoplasmic signaling domain comprising a functional signaling domain derived from a co-stimulatory molecule and a functional signaling domain derived from a stimulatory molecule. In one aspect, the CAR comprises a chimeric fusion protein comprising an extracellular antigen recognition domain, a transmembrane domain and an intracellular signaling domain comprising two functional signaling domains derived from one or more co-stimulatory molecule(s) and a functional signaling domain derived from a stimulatory molecule. In one aspect, the CAR comprises a chimeric fusion protein comprising an extracellular antigen recognition domain, a transmembrane domain and an intracellular signaling domain comprising at least two functional signaling domains derived from one or more co-stimulatory molecule(s) and a functional signaling domain derived from a stimulatory molecule. In one aspect the CAR comprises an optional leader sequence at the amino-terminus (N-ter) of the CAR fusion protein. In one aspect, the CAR further comprises a leader sequence at the N-terminus of the extracellular antigen recognition domain, wherein the leader sequence is optionally cleaved from the scFv domain during cellular processing and localization of the CAR to the cellular membrane. As used herein, the terms intracellular and cytoplasmic are used interchangeably.

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to encode polypeptides that elicit the desired immune response. Moreover, a skilled artisan will understand that an antigen need not be encoded by a “gene” at all. It is readily apparent that an antigen can be generated, synthesized or can be derived from a biological sample, or it can be a macromolecule that is not necessarily a polypeptide. Such a biological sample can include, but is not limited to a tissue sample, a tumor sample, a cell or a fluid with other biological components.

The term “anti-tumor effect” as used herein, refers to a biological effect which can be manifested by various means, including but not limited to, e.g., a decrease in tumor volume, a decrease in the number of tumor cells, a decrease in the number of metastases, an increase in life expectancy, decrease in tumor cell proliferation, decrease in tumor cell survival, or amelioration of various physiological symptoms associated with the cancerous condition. An “anti-tumor effect” can also be manifested by the ability of the peptides, polynucleotides, cells and antibodies of the invention in prevention of the occurrence of tumor in the first place.

As used herein, the term “autologous” is meant to refer to any material derived from the same individual to whom it is later to be re-introduced.

“Allogeneic” refers to any material derived from a different animal of the same species as the individual to whom the material is introduced. Two or more individuals are said to be allogeneic to one another when the genes at one or more loci are not identical. In some aspects, allogeneic material from individuals of the same species may be sufficiently unlike genetically to interact antigenically.

“Xenogeneic” refers to a graft derived from an animal of a different species.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

As used herein, the term “conservative sequence modifications” is intended to refer to amino acid modifications that do not significantly affect or alter the binding characteristics of the antibody or antibody fragment containing the amino acid sequence. Such conservative modifications include amino acid substitutions, additions and deletions. Modifications can be introduced into an antibody or antibody fragment of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody or antibody fragment of the invention can be replaced with other amino acid residues from the same side chain family and the altered antibody or antibody fragment can be tested for the ability to bind CD123 using the functional assays described herein.

By the term “stimulation,” is meant a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-β, and/or reorganization of cytoskeletal structures, and the like.

A “stimulatory molecule,” as the term is used herein, means a molecule expressed by a T cell that provide the primary cytoplasmic signaling sequence(s) that regulate primary activation of the TCR complex in a stimulatory way for at least some aspect of the T cell signaling pathway. In one aspect, the primary signal is initiated by, for instance, binding of a TCR/CD3 complex with an MHC molecule loaded with peptide, and which leads to mediation of a T cell response, including, but not limited to, proliferation, activation, differentiation, and the like. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences that are of particular use in the invention include those derived from TCR zeta, FcR gamma, FcR beta, CD3 gamma, CD3 delta, CD3 epsilon, CD5, CD22, CD79a, CD79b, CD278 (also known as “ICOS”) and CD66d. In one embodiment, the cytoplasmic signaling molecule in any one or more CARs of the invention comprises a cytoplasmic signaling sequence derived from CD3-zeta. In one embodiment, the cytoplasmic signaling sequence derived from CD3-zeta is the human sequence, or the equivalent residues from a non-human species, e.g., mouse, rodent, monkey, ape and the like.

An “antigen presenting cell” or “APC” as used herein, means an immune system cell such as an accessory cell (e.g., a B-cell, a dendritic cell, and the like) that displays foreign antigens complexed with major histocompatibility complexes (MHC's) on their surfaces. T-cells may recognize these complexes using their T-cell receptors (TCRs). APCs process antigens and present them to T-cells.

As used herein “zeta” or alternatively “zeta chain”, “CD3-zeta” or “TCR-zeta” is defined as the protein provided as GenBan accno. BAG36664.1, or the equivalent residues from a non-human species, e.g., mouse, rodent, monkey, ape and the like, and a “zeta stimulatory domain” or alternatively a “CD3-zeta stimulatory domain” or a “TCR-zeta stimulatory domain” is defined as the amino acid residues from the cytoplamic domain of the zeta chain that are sufficient to functionally transmit an initial signal necessary for T cell activation. In one aspect the cytoplasmic domain of zeta comprises residues 52 through 164 of GenBank accno. BAG36664.1 or the equivalent residues from a non-human species, e.g., mouse, rodent, monkey, ape and the like, that are functional orthologs thereof.

A “costimulatory molecule” refers to the cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that are required for an efficient immune response. Costimulatory molecules include, but are not limited to an MHC class I molecule, BTLA and a Toll ligand receptor, as well as OX40, CD27, CD28, CDS, ICAM-1, LFA-1 (CD11a/CD18) and 4-1BB (CD137).

As used herein “4-1BB” is defined as member of the TNFR superfamily with an amino acid sequence provided as GenBank accno. AAA62478.2, or the equivalent residues from a non-human species, e.g., mouse, rodent, monkey, ape and the like; and a “4-1BB costimulatory domain” are defined amino acid residues 214-255 of GenBank accno. AAA62478.2, or the equivalent residues from a non-human species, e.g., mouse, rodent, monkey, ape and the like. In one aspect, the “4-1BB costimulatory domain” is the human sequence or the equivalent residues from a non-human species, e.g., mouse, rodent, monkey, ape and the like. “Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene, cDNA, or RNA encodes a protein if transcription and translation of mRNA corresponding to that gene, cDNA, or RNA produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins or a RNA may also include introns to the extent that the nucleotide sequence encoding the protein may in some version contain an intron(s).

“Effective amount” or “therapeutically effective amount” are used interchangeably herein, and refer to an amount of a compound, formulation, material, or composition, as described herein effective to achieve a particular biological result. Such results may include, but are not limited to, the inhibition of virus infection as determined by any means suitable in the art.

As used herein “endogenous” refers to any material from or produced inside an organism, cell, tissue or system.

As used herein, the term “exogenous” refers to any material introduced from or produced outside an organism, cell, tissue or system.

The term “expression” as used herein is defined as the transcription and/or translation of a particular nucleotide sequence driven by its regulatory sequences.

A “transfer vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “transfer vector” includes an autonomously replicating plasmid or a virus. The term should also be construed to further include non-plasmid and non-viral compounds which facilitate transfer of nucleic acid into cells, such as, for example, polylysine compounds, liposomes, and the like. Examples of viral transfer vectors include, but are not limited to, adenoviral vectors, adeno-associated virus vectors, retroviral vectors, lentiviral vectors, and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

A “lentiviral vector” is a vector derived from at least a portion of a lentivirus genome, including especially a self-inactivating lentiviral vector as provided in Milone et al., Mol. Ther. 17 (8): 1453-1464 (2009). Other Examples or lentivirus vectors that may be used in the clinic as an alternative to the pELPS vector, include but not limited to, e.g., the LENTIVECTOR® gene delivery technology from Oxford BioMedica, the LENTIMAX™ vector system from Lentigen and the like. Nonclinical types of lentiviral vectors are also available and would be known to one skilled in the art.

The term “operably linked” or alternatively “transcriptional control” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Operably linked DNA sequences can be contiguous with each other and, where necessary to join two protein coding regions, are in the same reading frame.

“Parenteral” administration of an immunogenic composition includes, e.g., subcutaneous (s.c.), intravenous (i.v.), intramuscular (i.m.), or intrasternal injection, or infusion techniques.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

A “flexible polypeptide linker” as used in the context of an scFv refers to a peptide linker that consists of amino acids such as glycine and serine residues used alone or in combination, to link variable heavy and variable light chain regions together. In one embodiment, the flexible polypeptide linker is a Gly/Ser linker and comprises the amino acid sequence (Gly-Gly-Gly-Ser) n, where n is a positive integer equal to or greater than 1. For example, n=1, n=2, n=3, n=4, n=5 and n=6, n=7, n=8, n=9 and n=10. In one embodiment, the flexible polypeptide linkers include, but are not limited to, (Gly₄Ser)₄ or (Gly₄Ser)₃ In another embodiment, the linkers include multiple repeats of (Gly₂Ser), (GlySer) or (Gly₃Ser). Also included within the scope of the invention are linkers described in WO2012/138475, incorporated herein by reference in its entirety).

A “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the membrane of a cell.

The term “subject” is intended to include living organisms in which an immune response can be elicited (e.g., mammals including human).

As used herein, a “substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some aspects, the cells are cultured in vitro. In other aspects, the cells are not cultured in vitro.

By the term “synthetic” as it refers to a nucleic acid or polypeptide, including an antibody, is meant a nucleic acid, polypeptide, including an antibody, which has been generated by a mechanism not found naturally within a cell. In some instances, the term “synthetic” may include and therefore overlap with the term “recombinant” and in other instances, the term “synthetic” means that the nucleic acid, polypeptide, including an antibody, has been generated by purely chemical or other means.

The term “therapeutic” as used herein means a treatment. A therapeutic effect is obtained by reduction, suppression, remission, or eradication of a disease state.

The term “prophylaxis” as used herein means the prevention of or protective treatment for a disease or disease state.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The phrase “under transcriptional control” or “operatively linked” as used herein means that the promoter is in the correct location and orientation in relation to a polynucleotide to control the initiation of transcription by RNA polymerase and expression of the polynucleotide.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses.

By the term “specifically binds,” as used herein, is meant an antibody or antigen binding fragment thereof, or a ligand, which recognizes and binds with a cognate binding partner (e.g., a stimulatory and/or costimulatory molecule present on a T cell) protein present in a sample, but which antibody, antigen binding fragment thereof or ligand does not substantially recognize or bind other molecules in the sample.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

Adoptive Cell Transfer (ACT) using lymphocytes genetically engineered to express tumor-specific T cell receptor (TCR) or Chimeric Antigen Receptor (CAR) demonstrated high (50% to 90%) remission rates in patients with advanced (stage IV) cancers. However, several drawbacks including inability to rapidly alter treatment regimen, off- and on-target toxicities, and high costs of the recombinant T cell production hamper the advancement of this modality to general practice.

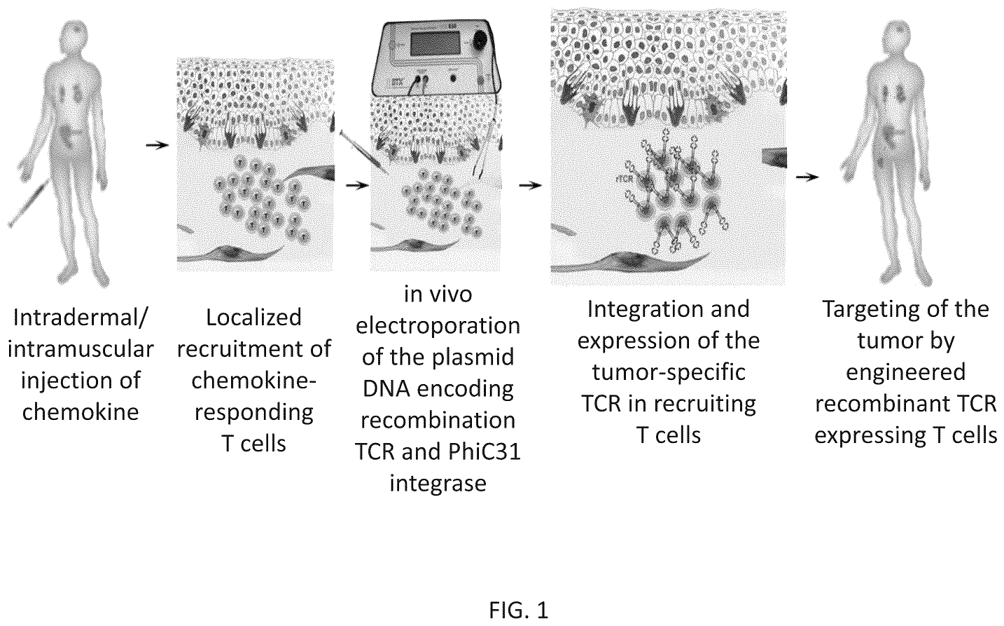

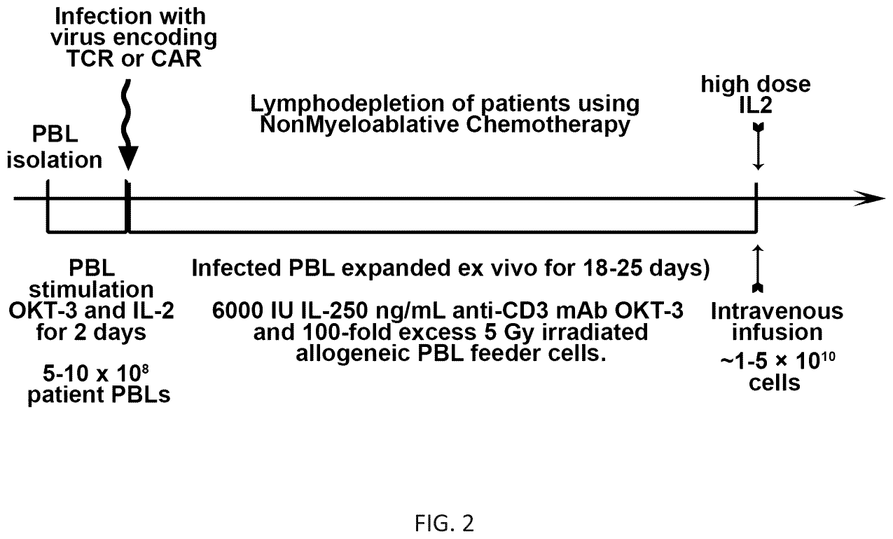

To address an unmet need to establish universal, generic and cost-effective modality and bring recombinant T cell-based vaccines to general public, the presently described invention utilizes a novel approach that allows intratumoral and/or intradermal generation of the tumor-specific recombinant T cells directly in a subject. In certain embodiments, this strategy involves: (i) chemokine-mediated recruitment of the T cells (total population or pre-defined T cell sub-sets) to a specific location (skin or tumor) via injection of the recombinant chemokine or electroporation of the chemokine-encoding plasmid, (ii) in vivo transfer of one or more plasmid DNA (mammalian expression vector) encoding a desired recombinant TCR or CAR and ΦC31 integrase (using e.g., in vivo electroporation or other efficient method of in vivo plasmid DNA delivery). After in vivo gene transfer, the genetic material encoding the desired TCR or CAR integrates into the genome of the chemokine-recruited T cells by means of ΦC31 mediated integration, which leads to generation of recombinant, antigen-specific T cells and T cell-mediated targeting of the antigen (e.g., tumor antigen) (FIG. 1).

As described herein, the method of the in vivo T cell genetic engineering is designed to overcome limitations of the current recombinant T cell-based approach in cancer immunotherapy: Use of plasmid DNA for the direct treatment of a cancer patient eliminates the necessity of the ex vivo manipulation with patient derived T cells that require GMP tissue culture facilities and specifically trained personnel leading to substantial (by preliminary estimates −100 times or more) reduction the overall treatment cost.

The method is further advantageous as it allows multiple treatments, rapid change in treatment regimen, concurrent and/or sequential use of various tumor-targeting TCR or CAR designed for different tumor-specific antigens.

The method also advances treatment by allowing for localized in-patient generation of the recombinant T cells allows avoiding systemic infusion of large quantities of the recombinant T cells and associated of-target toxicities including so-called “cytokine storm” caused by the infusion of a large number of the activated T cells into systemic compartment. It also, potentially, minimizes on-target toxicity.

Finally, localized pre-conditioning of the skin or tumor with chemokine(s) allows recruitment of the specific sets of T cells (pre-conditioning with CCL21 leads to the recruitment of the naive and central memory T cells). This T cell population is superior to others as it can give rise to the effector, tumor-targeting T cells and to T cells that generate immunologic memory. The latter could serve as a renewable source of the effector cells for continuous tumor targeting.

Another advantage of the present invention is that the strategy described herein is flexible and versatile with regard to type of TCR or CAR utilized. At present, a rather large number different tumor-specific TCR or CAR have been cloned and tested in pre-clinical and/or clinical studies for immunotargeting of various cancers. In the presently described method of the in vivo T cell genetic engineering, sequences encoding a desired TCR or CAR directed against an antigen, such as a tumor-specific antigen or tumor-associated antigen, can be incorporated into a plasmid or expression vector and administered in vivo to a subject in need of an immune response directed against the antigen. In certain embodiments, the in vivo administration of the TCR or CAR-encoding plasmid is done using a physical method of in vivo plasmid delivery, including but not limited to electroporation. However, the present invention is not limited to any particular form of in vivo plasmid delivery, and encompasses any of the various forms of in vivo plasmid delivery known to those of ordinary skill in the art.

For example, in one aspect, the present invention provides a method of inducing an immune response against a cancer antigen comprising: (i) recruitment of a subject's T cells or pre-defined T cell sub-sets to a specific location via injection of the recombinant chemokine or electroporation of the chemokine-encoding plasmid, (ii) in vivo transfer of one or more plasmids encoding recombinant TCR or CAR and an integrase (e.g., C31 integrase) via in vivo electroporation (or other efficient method of in vivo plasmid DNA delivery), wherein, after in vivo gene transfer, genetic material encoding the TCR or CAR integrates into the genome of the chemokine-recruited T cells by means of integrase (e.g., C31 integrase) mediated integration and; finally, generation of recombinant, tumor-specific T cells and T cell-mediated targeting of the tumor. Therefore, the tumor-specific T cells are targeted directly at the tumor site.

Treatment of a cancer patient could be done in the outpatient office, in hospital settings, during surgery on the unresectable lesions or, even in the field. In one embodiment, the skin or a tumor lesion site is pre-treated/pre-conditioned via electroporation of a plasmid encoding secondary lymphoid chemokine (e.g., CCL21) or via injection of the recombinant protein. In one embodiment, 24 to 48 hours later the chemokine primed site is treated via electroporation of one or more plasmids encoding tumor-specific receptor (TCR or CAR) and integrase (e.g., ΦC31 integrase). In certain embodiments the steps of T-cell recruitment and/or administration of one or more plasmids encoding the TCR, CAR and/or integrase, are repeated multiple times to achieve clinically relevant response.

The methods of the invention allow (i) use of a subject's immune system without lymphoablation; (ii) multiple concurrent or consequent treatments to achieve sufficient number of recombinant T cells to complete immune-mediated remission of the malignant lesions in outpatient setting; (iii) targeting of different tumor-associated antigens via recombinant TCR and CAR; (iv) rapid alteration of the treatment regimen; (v) substantial (estimated 100 fold) reduction of the treatment cost as compared to ACT making it affordable and available for general patient population; (vi) treatment of various types of cancer to which recombinant TCR or CAR are developed; and (vii) reduction of on- and off-target toxicities associated with the infusion of a large number of activated recombinant T cells in ACT. Further, the presently described technology could be used as an investigative tool to rapidly assess the efficacy of the tumor-targeting TCR and CAR in settings of established tumor lesions.

### Cytokine Composition

In some aspects, the present invention provides a cytokine composition comprising one or more agents that recruit T cells or T cell subsets to a site in which the composition is administered. In one embodiment, the cytokine comprises an agent capable of recruiting one or more naïve T cells to the site of administration.

In some aspects, the cytokine composition comprises at least one chemokine ligand or a nucleic acid molecule encoding at least one chemokine ligand. In one embodiment, the chemokine ligand is a ligand for one or more of CCR3, CCR4, CCR8, CXCR4, CCR5, CCR7, CXCR3, or CXCR6 chemokine receptors. In one embodiment, the chemokine ligand is one or more of CCL2, CCL3, CCL4, CCL5, macrophage inflammatory proteins (MIP-1α), CXCL9, CXCL10, CXCL12, CXCL16, CCL17, CCL19, CCL20, CCL21, CCL22, or CCL27.

In one embodiment, the cytokine composition comprises a combination of CCL5 and CCL22. In one embodiment, the cytokine composition comprises a combination of CCL21 and CCL22. In one embodiment, the cytokine composition comprises CCL2. In one embodiment, the cytokine composition comprises CCL21.

In one embodiment, the cytokine composition comprises a combination of a nucleic acid molecule encoding CCL5 and a nucleic acid molecule encoding CCL22. In one embodiment, the cytokine composition comprises a combination of a nucleic acid molecule encoding CCL21 and a nucleic acid molecule encoding CCL22. In one embodiment, the cytokine composition comprises a nucleic acid molecule encoding CCL2. In one embodiment, the cytokine composition comprises a nucleic acid molecule encoding CCL21.

In one embodiment, the composition may further comprise one or more additional agent to increase the level of T cell recruitment. Exemplary additional agents for increasing T cell recruitment include, but are not limited to, IFN-γ, IFN-γ, granzyme B, perforin and inducible T cell co-stimulator (ICOS).

In some aspects, the method of the invention includes administering a cytokine composition comprising one or more agents for recruiting T cells or T cell subsets to the site of administration, whereby T cells or T cell subsets become locally concentrated. In some embodiments, the cytokine comprises an agent capable of recruiting one or more naïve T cells to the site of administration.

In certain embodiments, a specific cytokine composition is administered to induce recruitment of specific types of T cells. For example, in one embodiment, CCL21 or a nucleic acid molecule encoding CCL21 is administered to preferentially recruit CCR7+ naïve T-cells and T_{CM} to the administration site. In one embodiment, CCL17 or a nucleic acid molecule encoding CCL17 is administered to preferentially recruit peripheral memory and effector CCR4+ T cells to the administration site. In one embodiment, CCL22 or a nucleic acid molecule encoding CCL22 is administered to preferentially recruit peripheral memory and effector CCR4+ T cells to the administration site. In one embodiment, CCL27 or a nucleic acid molecule encoding CCL27 is administered to preferentially recruit CCR10+ T helper (Th) cells to the administration site. In one embodiment, CCL5 or a nucleic acid molecule encoding CCL5 is administered to preferentially recruit CCR4 and CCR5 CD4+ Th1 and CD8+ cytotoxic lymphocytes (CTL) to the administration site.

In certain embodiments, the cytokine composition is administered locally to a desired site of the subject. In certain embodiments, the cytokine composition is administered intradermally, intratumorally, intranodally, subcutaneously, intramuscularly, or intramedullary.

In certain embodiments, the administration of the cytokine composition is repeated one or more times to enhance T cell recruitment. In one embodiment, the administration of the cytokine composition is repeated one or more times prior to subsequent administration of TCR or CAR-encoding nucleic acid molecules. In one embodiment, the administration of the cytokine composition is repeated one or more times after administration of TCR or CAR-encoding nucleic acid molecules.

In one embodiment, the administration of the cytokine composition is repeated every day, every 2 days, every 3 days, every 4 days, every 5 days, every 6 days, every 7 days, every 8 days, every 9 days, every 10 days, every 11 days, every 12 days, every 13 days, or every 14 days. In one embodiment, the administration of the cytokine composition is repeated every week, every 2 weeks, every 3 weeks, every 4 weeks, every 5 weeks, every 6 weeks, every 7 weeks, every 8 weeks, every 9 weeks, every 10 weeks, every 11 weeks, or every 12 weeks. In one embodiment, the administration of the cytokine composition is repeated every month, every 2 months, every 3 months, every 4 months, every 5 months, every 6 months, every 7 months, every 8 months, every 9 months, every 10 months, every 11 months, or every 12 months.

In one embodiment, the cytokine composition may be administered to deliver a dose of between 1 ng/kg and 100 mg/kg per administration. In one embodiment, the cytokine composition may be administered to deliver a dose of between 1 ng/kg and 500 mg/kg per administration.

### Antigen Receptor Composition

In one aspect, the present invention provides an antigen receptor composition for genetically engineering T cells in vivo. In one embodiment, the antigen receptor composition comprises a nucleic acid molecule encoding an antigen receptor.

In one embodiment, the antigen receptor is or includes a T cell receptor (TCR), such as a high-affinity TCR, or functional non-TCR antigen receptor, such as a chimeric antigen receptor (CAR). In some aspects, the receptor specifically binds to an antigen expressed by cells of a disease or condition to be treated.

The antigen receptor of the invention can be generated to be reactive to any desirable antigen of interest, or fragment thereof, including, but not limited to a tumor antigen, a bacterial antigen, a viral antigen or a self-antigen. In the context of the present invention, “tumor antigen” or “hyperporoliferative disorder antigen” or “antigen associated with a hyperproliferative disorder,” refers to antigens that are common to specific hyperproliferative disorders such as cancer. In certain aspects, the hyperproliferative disorder antigens of the present invention are derived from cancers including, but not limited to, primary or metastatic melanoma, mesothelioma, thymoma, lymphoma, sarcoma, lung cancer, liver cancer, non-Hodgkin's lymphoma, Hodgkins lymphoma, leukemias, uterine cancer, cervical cancer, bladder cancer, kidney cancer and adenocarcinomas such as breast cancer, prostate cancer, ovarian cancer, pancreatic cancer, and the like.

The antigens discussed herein are merely included by way of example. The list is not intended to be exclusive and further examples will be readily apparent to those of skill in the art.

Tumor antigens are proteins that are produced by tumor cells that elicit an immune response. The selection of the antigen binding domain of the invention will depend on the particular type of cancer to be treated. Tumor antigens are well known in the art and include, for example, a glioma-associated antigen, carcinoembryonic antigen (CEA), β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin. Another exemplary tumor antigen is chondroitin sulfate proteoglycan 4 (CSPG4) (also referred to as melanoma-associated chondroitin sulfate proteoglycan (MCSP), high-molecular-weight melanoma-associated antigen (HMW-MAA), or neuron-glial antigen 2 (NG2)).

In one embodiment, the tumor antigen comprises one or more antigenic cancer epitopes associated with a malignant tumor. Malignant tumors express a number of proteins that can serve as target antigens for an immune attack. These molecules include but are not limited to tissue-specific antigens such as MART-1, tyrosinase and GP 100 in melanoma and prostatic acid phosphatase (PAP) and prostate-specific antigen (PSA) in prostate cancer. Other target molecules belong to the group of transformation-related molecules such as the oncogene HER-2/Neu/ErbB-2. Yet another group of target antigens are onco-fetal antigens such as carcinoembryonic antigen (CEA). In B-cell lymphoma the tumor-specific idiotype immunoglobulin constitutes a truly tumor-specific immunoglobulin antigen that is unique to the individual tumor. B-cell differentiation antigens such as CD19, CD20 and CD37 are other candidates for target antigens in B-cell lymphoma. Some of these antigens (CEA, HER-2, CD19, CD20, idiotype) have been used as targets for passive immunotherapy with monoclonal antibodies with limited success.

The type of tumor antigen referred to in the invention may also be a tumor-specific antigen (TSA) or a tumor-associated antigen (TAA). A TSA is unique to tumor cells and does not occur on other cells in the body. A TAA associated antigen is not unique to a tumor cell and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development when the immune system is immature and unable to respond or they may be antigens that are normally present at extremely low levels on normal cells but which are expressed at much higher levels on tumor cells.

Non-limiting examples of TSA or TAA antigens include the following: Differentiation antigens such as MART-1/MelanA (MART-I), gp100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185crbB2, p180erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS1, SDCCAG16, TA-90\Mac-2 binding protein\cyclophilin C-associated protein, TAAL6, TAG72, TLP, and TPS.

In certain embodiments, the antigen receptor (e.g., the TCR or CAR) targets an antigen that includes but is not limited to CD19, tyrosinase, CSPG4, CD20, CD22, ROR1, Mesothelin, CD33/IL3Ra, c-Met, PSMA, Glycolipid F77, EGFRvIII, GD-2, MY-ESO-1 TCR, MAGE A3 TCR, and the like.

Depending on the desired antigen to be targeted, the antigen receptor can be engineered to include the appropriate antigen binding moiety that is specific to the desired antigen target. For example, if CD19 is the desired antigen that is to be targeted, an antibody for CD19 can be used as the antigen binding moiety for incorporation into antigen receptor.

In certain embodiments, the antigen receptor is a TCR. A TCR is a disulfide-linked heterodimeric protein consisting of two variable chains expressed as part of a complex with the invariant CD3 chain molecules. A TCR is found on the surface of T cells, and is responsible for recognizing antigens as peptides bound to major histocompatibility complex (MHC) molecules. In certain embodiments, a TCR comprises an alpha chain and a beta chain (encoded by TRA and TRB, respectively). In certain embodiments, a TCR comprises a gamma chain and a delta chain (encoded by TRG and TRD, respectively).

Each chain of a TCR is composed of two extracellular domains: Variable (V) region and a Constant (C) region. The Constant region is proximal to the cell membrane, followed by a transmembrane region and a short cytoplasmic tail. The Variable region binds to the peptide/MHC complex. The variable domain of both chains each has three complementarity determining regions (CDRs).

In certain embodiments, a TCR can form a receptor complex with three dimeric signaling modules CD3δ/ε, CD3γ/ε and CD247 ζ/ζ or ζ/η. When a TCR complex engages with its antigen and MHC (peptide/MHC), the T cell expressing the TCR complex is activated.

In one embodiment, the TCR is a recombinant TCR. In certain embodiments, the TCR is a naturally occurring TCR. In certain embodiments, the TCR is a non-naturally occurring TCR. In certain embodiments, the TCR differs from any naturally occurring TCR by at least one amino acid residue. In certain embodiments, the TCR is modified from a naturally occurring TCR by at least one amino acid residue.

In certain embodiments, the TCR differs from any naturally occurring TCR by at least 2, 3, 4, 5, 6, 7, 8, 9, 10, 11, 12, 13, 14, 15, 20, 25, 30, 40, 50, 60, 70, 80, 90, 100 or more amino acid residues. In certain embodiments, the TCR is modified from a naturally occurring TCR by at least one amino acid residue. In certain embodiments, the TCR is modified from a naturally occurring TCR by at least 2, 3, 4, 5, 6, 7, 8, 9, 10, 11, 12, 13, 14, 15, 20, 25, 30, 40, 50, 60, 70, 80, 90, 100 or more amino acid residues.

In certain embodiments, the TCR comprises one or more mutations, relative to a naturally occurring TCR, in the constant region, variable region, a CDR, transmembrane domain, or cytoplasmic domain.

In certain embodiments, the TCR is modified to comprise one or more intracellular signaling domains. For example, in one embodiment, the TCR is modified to comprise one or more primary cytoplasmic signaling sequences, such as ITAMs. Examples of ITAM containing primary cytoplasmic signaling sequences that are of particular use in the invention include those derived from TCR zeta, FcR gamma, FcR beta, CD3 zeta, CD3 gamma, CD3 delta, CD3 epsilon, CD5, CD22, CD79a, CD79b, and CD66d. In one embodiment, the TCR is modified to comprise one or more costimulatory signaling regions, such as an intracellular domain of a costimulatory molecule. Examples of such molecules include CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, and a ligand that specifically binds with CD83, and the like. In one embodiment, the TCR is modified to comprising the CD3 zeta, CD137 (4-1BB) and CD28 signaling domains.

In one embodiment, the CAR contains an extracellular antigen-binding domain. In one embodiment, the CAR comprises a transmembrane domain. In one embodiment, the CAR comprises a cytoplasmic domain, or otherwise an intracellular signaling domain.

The extracellular domain may be obtained from any of the wide variety of extracellular domains or secreted proteins associated with ligand binding and/or signal transduction. In one embodiment, the extracellular domain may consist of an Ig heavy chain which may in turn be covalently associated with Ig light chain by virtue of the presence of CH1 and hinge regions, or may become covalently associated with other Ig heavy/light chain complexes by virtue of the presence of hinge, CH2 and CH3 domains. In the latter case, the heavy/light chain complex that becomes joined to the chimeric construct may constitute an antibody with a specificity distinct from the antibody specificity of the chimeric construct. Depending on the function of the antibody, the desired structure and the signal transduction, the entire chain may be used or a truncated chain may be used, where all or a part of the CH1, CH2, or CH3 domains may be removed or all or part of the hinge region may be removed.

The extracellular domain can be directed to any desired antigen. For example, when an antitumor CAR is desired, the extracellular domain chosen to be incorporated into the CAR can be an antigen that is associated with the tumor. The tumor may be any type of tumor as long as it has a cell surface antigen which is recognized by the CAR. In another embodiment, the CAR may one for which a specific monoclonal antibody currently exists or can be generated in the future.

In one embodiment, the CAR comprises a target-specific binding element otherwise referred to as an antigen binding domain. The choice of moiety depends upon the type and number of ligands that define the surface of a target cell. For example, the antigen binding domain may be chosen to recognize a ligand that acts as a cell surface marker on target cells associated with a particular disease state. Thus examples of cell surface markers that may act as ligands for the antigen moiety domain in the CAR include those associated with viral, bacterial and parasitic infections, autoimmune disease and cancer cells.

With respect to the transmembrane domain, the CAR can be designed to comprise a transmembrane domain that is fused to the extracellular domain of the CAR. In one embodiment, the transmembrane domain that naturally is associated with one of the domains in the CAR is used. In some instances, the transmembrane domain can be selected or modified by amino acid substitution to avoid binding of such domains to the transmembrane domains of the same or different surface membrane proteins to minimize interactions with other members of the receptor complex.

The transmembrane domain may be derived either from a natural or from a synthetic source. Where the source is natural, the domain may be derived from any membrane-bound or transmembrane protein. Transmembrane regions of particular use in this invention may be derived from (i.e. comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154. Alternatively, the transmembrane domain may be synthetic, in which case it will comprise predominantly hydrophobic residues such as leucine and valine. In certain embodiments, a triplet of phenylalanine, tryptophan and valine will be found at each end of a synthetic transmembrane domain. Optionally, a short oligo- or polypeptide linker, for example between 2 and 10 amino acids in length, may form the linkage between the transmembrane domain and the cytoplasmic signaling domain of the CAR. A glycine-serine doublet provides a particularly suitable linker.

In some embodiments, the intracellular signaling domain of the CAR comprises an ITAM-containing sequence. In some embodiments, the intracellular signaling domain of the CAR comprises an intracellular signaling domain of a T cell costimulatory molecule.

The cytoplasmic domain or otherwise the intracellular signaling domain of the CAR is responsible for activation of at least one of the normal effector functions of the immune cell in which the CAR has been placed in. The term “effector function” refers to a specialized function of a cell. Effector function of a T cell, for example, may be cytolytic activity or helper activity including the secretion of cytokines. Thus the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

Exemplary intracellular signaling domains for use in the CAR include the cytoplasmic sequences of the T cell receptor (TCR) and co-receptors that act in concert to initiate signal transduction following antigen receptor engagement, as well as any derivative or variant of these sequences and any synthetic sequence that has the same functional capability.

It is known that, in certain instances, signals generated through the TCR alone are insufficient for full activation of the T cell and that a secondary or co-stimulatory signal is also required. Thus, T cell activation can be said to be mediated by two distinct classes of cytoplasmic signaling sequence: those that initiate antigen-dependent primary activation through the TCR (primary cytoplasmic signaling sequences) and those that act in an antigen-independent manner to provide a secondary or co-stimulatory signal (secondary cytoplasmic signaling sequences).

Primary cytoplasmic signaling sequences regulate primary activation of the TCR complex either in a stimulatory way, or in an inhibitory way. Primary cytoplasmic signaling sequences that act in a stimulatory manner may contain signaling motifs which are known as immunoreceptor tyrosine-based activation motifs or ITAMs.

Examples of ITAM containing primary cytoplasmic signaling sequences that are of particular use in the invention include those derived from TCR zeta, FcR gamma, FcR beta, CD3 zeta, CD3 gamma, CD3 delta, CD3 epsilon, CD5, CD22, CD79a, CD79b, and CD66d.

In one embodiment, the cytoplasmic domain of the CAR can be designed to comprise the CD3-zeta signaling domain by itself or combined with any other desired cytoplasmic domain(s) useful in the context of the CAR. For example, the cytoplasmic domain of the CAR can comprise a CD3 zeta chain portion and a costimulatory signaling region. The costimulatory signaling region refers to a portion of the CAR comprising the intracellular domain of a costimulatory molecule. A costimulatory molecule is a cell surface molecule other than an antigen receptor or their ligands that is required for an efficient response of lymphocytes to an antigen. Examples of such molecules include CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, and a ligand that specifically binds with CD83, and the like.

The cytoplasmic signaling sequences within the cytoplasmic signaling portion of the CAR may be linked to each other in a random or specified order. Optionally, a short oligo- or polypeptide linker, for example, between 2 and 10 amino acids in length may form the linkage. A glycine-serine doublet provides a particularly suitable linker.

In one embodiment, the antigen receptor composition comprises a nucleic acid molecule encoding an antigen receptor, such as a TCR or CAR. In certain embodiments, the method comprises the stable integration of the nucleic acid molecule, or portion thereof, encoding an antigen receptor into the DNA of a T cell of the subject. In one embodiment, the antigen receptor composition comprises a retroviral or lentiviral vector that allows for long-term gene transfer since they allow long-term, stable integration of a transgene and its propagation in daughter cells. In certain embodiments, the nucleic acid molecule comprises a recognition target site for interaction with a recombinase, to allow for integration of the nucleic acid molecule, or portion thereof, encoding the antigen receptor into the DNA of a T cell of subject mediated by an integration composition e co-administered to the subject, as described elsewhere herein. Exemplary recognition target sites include, but is not limited to, FRT, loxP, and attachment sites such as attB sites.

In some embodiments, the method comprises administration of one or more compositions for genetically engineering T cells in vivo. In one embodiment, the invention comprises administration of a nucleic acid molecule encoding an antigen receptor to the subject.

In one embodiment, the method comprises administering to a subject a composition comprising a recombinant nucleic acid molecule comprising a nucleic acid sequence encoding a CAR, wherein the CAR comprises an antibody fragment that binds specifically to an antigen. In one embodiment, the sequence of the antibody fragment is contiguous with and in the same reading frame as a nucleic acid sequence encoding an intracellular domain. In one embodiment, the intracellular domain or otherwise the cytoplasmic domain comprises, a costimulatory signaling region and/or a zeta chain portion. In one embodiment, the costimulatory signaling region refers to a portion of the CAR comprising the intracellular domain of a costimulatory molecule.

In one aspect, the composition comprises an isolated chimeric nucleic acid construct comprising sequences of a CAR, wherein the sequence comprises the nucleic acid sequence of an antigen binding domain operably linked to the nucleic acid sequence of an intracellular domain.

In certain embodiments, the antigen receptor composition is administered locally to a desired site of the subject. In certain embodiments, the antigen receptor composition is administered intradermally, intratumorally, intranodally, subcutaneously, intramuscularly, or intramedullary. In certain embodiments, the antigen receptor composition is administered at the same site, or substantially the same site, as the site in which the cytokine composition is administered, thereby efficiently genetically modifying the recruited T cells with the administered antigen receptor composition.

In certain embodiments, the administration of the antigen receptor composition is repeated one or more times to enhance therapeutic effect. In one embodiment, the administration of the antigen receptor composition is repeated one or more times after administration of the cytokine composition.

In one embodiment, the administration of the antigen receptor composition is repeated every day, every 2 days, every 3 days, every 4 days, every 5 days, every 6 days, every 7 days, every 8 days, every 9 days, every 10 days, every 11 days, every 12 days, every 13 days, or every 14 days. In one embodiment, the administration of the antigen receptor composition is repeated every week, every 2 weeks, every 3 weeks, every 4 weeks, every 5 weeks, every 6 weeks, every 7 weeks, every 8 weeks, every 9 weeks, every 10 weeks, every 11 weeks, or every 12 weeks. In one embodiment, the administration of the antigen receptor composition is repeated every month, every 2 months, every 3 months, every 4 months, every 5 months, every 6 months, every 7 months, every 8 months, every 9 months, every 10 months, every 11 months, or every 12 months.

In one embodiment, the antigen receptor composition may be administered to deliver a dose of between 1 ng/kg and 100 mg/kg per administration. In one embodiment, the antigen receptor composition may be administered to deliver a dose of between 1 ng/kg and 500 mg/kg per administration.

### Integration Composition

In one aspect, the present invention provides an integration composition comprising an agent that promotes integration or insertion of the nucleic acid molecule, or portion thereof, encoding the antigen receptor into T cells of the subject.

In certain embodiments, the integration composition comprises a recombinase or a nucleic acid molecule encoding a recombinase. The types of recombinases that can be administered in accordance with the methods of the invention include, but are not limited to, tyrosine recombinases, serine recombinases, bacteriophage integrase, tyrosine integrases, serine integrases, and the like. Specific recombinases that may be administered include, but are not limited to, ΦC31 integrase, Cre recombinase, Flp recombinase, Bxb1 integrase, and the like.

In one embodiment, the integration composition comprises a retroviral integrase or nucleic acid molecule encoding a retroviral integrase.

In one embodiment, the method of the invention further comprises administering an integration composition to the subject. In one embodiment, the integration composition promotes integration or insertion of the nucleic acid molecule, or portion thereof, encoding the antigen receptor into T cells of the subject.

In certain embodiments, the administration of the integration composition is repeated one or more times to enhance therapeutic effect. In one embodiment, the administration of the integration composition is repeated one or more times after administration of the antigen receptor composition.

In one embodiment, the administration of the integration composition is repeated every day, every 2 days, every 3 days, every 4 days, every 5 days, every 6 days, every 7 days, every 8 days, every 9 days, every 10 days, every 11 days, every 12 days, every 13 days, or every 14 days. In one embodiment, the administration of the integration composition is repeated every week, every 2 weeks, every 3 weeks, every 4 weeks, every 5 weeks, every 6 weeks, every 7 weeks, every 8 weeks, every 9 weeks, every 10 weeks, every 11 weeks, or every 12 weeks. In one embodiment, the administration of integration composition is repeated every month, every 2 months, every 3 months, every 4 months, every 5 months, every 6 months, every 7 months, every 8 months, every 9 months, every 10 months, every 11 months, or every 12 months.

In one embodiment, the integration composition may be administered to deliver a dose of between 1 ng/kg and 100 mg/kg per administration. In one embodiment, the integration composition may be administered to deliver a dose of between 1 ng/kg and 500 mg/kg per administration.

### Peptides

In certain aspects, one or more of the compositions described herein are peptides, proteins, or variants thereof. For example, in certain embodiments, the cytokine composition comprises a recombinant peptide, protein, or variant thereof. In certain embodiments, the recombinase comprises a recombinant peptide, protein, or variant thereof.

The peptide of the present invention may be made using chemical methods. For example, peptides can be synthesized by solid phase techniques (Roberge J Y et al (1995) Science 269:202-204), cleaved from the resin, and purified by preparative high performance liquid chromatography. Automated synthesis may be achieved, for example, using the ABI 431 A Peptide Synthesizer (Perkin Elmer) in accordance with the instructions provided by the manufacturer.

The invention should also be construed to include any form of a peptide having substantial homology to the peptides disclosed herein. In certain embodiments, a peptide which is “substantially homologous” is about 60% homologous, about 70% homologous, about 80% homologous, about 90% homologous, about 91% homologous, about 92% homologous, about 93% homologous, about 94% homologous, about 95% homologous, about 96% homologous, about 97% homologous, about 98% homologous, or about 99% homologous to amino acid sequence of the peptides disclosed herein.

The peptide may alternatively be made by recombinant means or by cleavage from a longer polypeptide. The composition of a peptide may be confirmed by amino acid analysis or sequencing.

The variants of the polypeptides according to the present invention may be (i) one in which one or more of the amino acid residues are substituted with a conserved or non-conserved amino acid residue and such substituted amino acid residue may or may not be one encoded by the genetic code, (ii) one in which there are one or more modified amino acid residues, e.g., residues that are modified by the attachment of substituent groups, (iii) one in which the polypeptide is an alternative splice variant of the polypeptide of the present invention, (iv) fragments of the polypeptides and/or (v) one in which the polypeptide is fused with another polypeptide, such as a leader or secretory sequence or a sequence which is employed for purification (for example, His-tag) or for detection (for example, Sv5 epitope tag). The fragments include polypeptides generated via proteolytic cleavage (including multi-site proteolysis) of an original sequence. Variants may be post-translationally, or chemically modified. Such variants are deemed to be within the scope of those skilled in the art from the teaching herein.

As known in the art the “similarity” between two polypeptides is determined by comparing the amino acid sequence and its conserved amino acid substitutes of one polypeptide to a sequence of a second polypeptide. Variants are defined to include polypeptide sequences different from the original sequence, for example different from the original sequence in less than 40% of residues per segment of interest, different from the original sequence in less than 25% of residues per segment of interest, different by less than 10% of residues per segment of interest, or different from the original protein sequence in just a few residues per segment of interest and at the same time sufficiently homologous to the original sequence to preserve the functionality of the original sequence and/or the ability to bind to ubiquitin or to a ubiquitylated protein. The present invention includes amino acid sequences that are at least 60%, 65%, 70%, 72%, 74%, 76%, 78%, 80%, 90%, 91%, 92%, 93%, 94%, 95%, 96%, 97%, 98%, or 99% similar or identical to the original amino acid sequence. The degree of identity between two polypeptides is determined using computer algorithms and methods that are widely known for the persons skilled in the art. In certain instances, the identity between two amino acid sequences is determined by using the BLASTP algorithm [BLAST Manual, Altschul, S., et al., NCBI NLM NIH Bethesda, Md. 20894, Altschul, S., et al., J. Mol. Biol. 215:403-410 (1990)].

The peptides of the invention can be post-translationally modified. For example, post-translational modifications that fall within the scope of the present invention include signal peptide cleavage, glycosylation, acetylation, isoprenylation, proteolysis, myristoylation, protein folding and proteolytic processing, etc. Some modifications or processing events require introduction of additional biological machinery. For example, processing events, such as signal peptide cleavage and core glycosylation, are examined by adding canine microsomal membranes or Xenopus egg extracts (U.S. Pat. No. 6,103,489) to a standard translation reaction.

The peptides of the invention may include unnatural amino acids formed by post-translational modification or by introducing unnatural amino acids during translation. A variety of approaches are available for introducing unnatural amino acids during protein translation. By way of example, special tRNAs, such as tRNAs which have suppressor properties, suppressor tRNAs, have been used in the process of site-directed non-native amino acid replacement (SNAAR). In SNAAR, a unique codon is required on the mRNA and the suppressor tRNA, acting to target a non-native amino acid to a unique site during the protein synthesis (described in WO90/05785). However, the suppressor tRNA must not be recognizable by the aminoacyl tRNA synthetases present in the protein translation system. In certain cases, a non-native amino acid can be formed after the tRNA molecule is aminoacylated using chemical reactions which specifically modify the native amino acid and do not significantly alter the functional activity of the aminoacylated tRNA. These reactions are referred to as post-aminoacylation modifications. For example, the epsilon-amino group of the lysine linked to its cognate tRNA (tRNALYS), could be modified with an amine specific photoaffinity label.

The term “functionally equivalent” as used herein refers to a peptide according to the invention that retains at least one biological function or activity of a wild-type cytokine or recombinase.

A peptide or protein of the invention may be conjugated with other molecules, such as proteins, to prepare fusion proteins. This may be accomplished, for example, by the synthesis of N-terminal or C-terminal fusion proteins provided that the resulting fusion protein retains the functionality of the wild-type cytokine or integrase comprising peptide.

A peptide or protein of the invention may be phosphorylated using conventional methods such as the method described in Reedijk et al. (The EMBO Journal 11 (4): 1365, 1992).

Cyclic derivatives of the peptides of the invention are also part of the present invention. Cyclization may allow the peptide to assume a more favorable conformation for association with other molecules. Cyclization may be achieved using techniques known in the art. For example, disulfide bonds may be formed between two appropriately spaced components having free sulfhydryl groups, or an amide bond may be formed between an amino group of one component and a carboxyl group of another component. Cyclization may also be achieved using an azobenzene-containing amino acid as described by Ulysse, L., et al., J. Am. Chem. Soc. 1995, 117, 8466-8467. The components that form the bonds may be side chains of amino acids, non-amino acid components or a combination of the two. In an embodiment of the invention, cyclic peptides may comprise a beta-turn in the right position. Beta-turns may be introduced into the peptides of the invention by adding the amino acids Pro-Gly at the right position.

It may be desirable to produce a cyclic peptide which is more flexible than the cyclic peptides containing peptide bond linkages as described above. A more flexible peptide may be prepared by introducing cysteines at the right and left position of the peptide and forming a disulphide bridge between the two cysteines. The two cysteines are arranged so as not to deform the beta-sheet and turn. The peptide is more flexible as a result of the length of the disulfide linkage and the smaller number of hydrogen bonds in the beta-sheet portion. The relative flexibility of a cyclic peptide can be determined by molecular dynamics simulations.

In a particular embodiment of the invention, the peptide of the invention further comprises the amino acid sequence of a tag. The tag includes but is not limited to: polyhistidine tags (His-tags) (for example H6 and H10, etc.) or other tags for use in IMAC systems, for example, Ni²⁺ affinity columns, etc., GST fusions, MBP fusions, streptavidine-tags, the BSP biotinylation target sequence of the bacterial enzyme BIRA and tag epitopes that are directed by antibodies (for example c-myc tags, FLAG-tags, among others). As will be observed by a person skilled in the art, the tag peptide can be used for purification, inspection, selection and/or visualization of the fusion protein of the invention. In a particular embodiment of the invention, the tag is a detection tag and/or a purification tag. It will be appreciated that the tag sequence will not interfere in the function of the protein of the invention.

Accordingly, the peptides of the invention can be fused to another peptide or tag, such as a leader or secretory sequence or a sequence which is employed for purification or for detection. In a particular embodiment, the peptide of the invention comprises the glutathione-S-transferase protein tag which provides the basis for rapid high-affinity purification of the polypeptide of the invention. Indeed, this GST-fusion protein can then be purified from cells via its high affinity for glutathione. Agarose beads can be coupled to glutathione, and such glutathione-agarose beads bind GST-proteins. Thus, in a particular embodiment of the invention, the peptide of the invention is bound to a solid support. In one embodiment, if the peptide of the invention comprises a GST moiety, the polypeptide is coupled to a glutathione-modified support. In a particular case, the glutathione modified support is a glutathione-agarose bead. Additionally, a sequence encoding a protease cleavage site can be included between the affinity tag and the peptide sequence, thus permitting the removal of the binding tag after incubation with this specific enzyme and thus facilitating the purification of the corresponding protein of interest.

The invention also relates to peptides comprising a cytokine or recombinase fused to, or integrated into, a target protein, and/or a targeting domain capable of directing the chimeric protein to a desired cellular component or cell type or tissue. The chimeric proteins may also contain additional amino acid sequences or domains. The chimeric proteins are recombinant in the sense that the various components are from different sources, and as such are not found together in nature (i.e. are heterologous).

In one embodiment, a target protein may be a protein that is mutated or over expressed in a disease or condition. In one embodiment, the target protein is underexpressed in a disease or condition. The targeting domain can be a membrane spanning domain, a membrane binding domain, or a sequence directing the protein to associate with for example vesicles or with the nucleus. The targeting domain can target a peptide to a particular cell type or tissue. For example, the targeting domain can be a cell surface ligand or an antibody against cell surface antigens of a target tissue (e.g. tumor tissue). A targeting domain may target the peptide of the invention to a cellular component.

Combined with certain formulations, such peptides can be effective intracellular agents. However, in order to increase the efficacy of such peptides, the peptide of the invention can be provided a fusion peptide along with a second peptide which promotes “transcytosis”, e.g., uptake of the peptide by epithelial cells. To illustrate, the integrase peptide of the present invention can be provided as part of a fusion polypeptide with all or a fragment of the N-terminal domain of the HIV protein Tat, e.g., residues 1-72 of Tat or a smaller fragment thereof which can promote transcytosis. In other embodiments, the integrase peptide of the present invention can be provided as part of a fusion polypeptide with all or a portion of the antenopedia III protein.

To further illustrate, the peptide of the invention can be provided as a chimeric peptide which includes a heterologous peptide sequence (“internalizing peptide”) which drives the translocation of an extracellular form of the peptide across a cell membrane in order to facilitate intracellular localization of the peptide. In this regard, the peptide is one which is active intracellularly. The internalizing peptide, by itself, is capable of crossing a cellular membrane by, e.g., transcytosis, at a relatively high rate. The internalizing peptide is conjugated, e.g., as a fusion protein, to a peptide comprising wild-type integrase. The resulting chimeric peptide is transported into cells at a higher rate relative to the peptide alone to thereby provide a means for enhancing its introduction into cells to which it is applied.

In other embodiments, the subject compositions are peptidomimetics of the peptide of the invention. Peptidomimetics are compounds based on, or derived from, peptides and proteins. The peptidomimetics of the present invention typically can be obtained by structural modification of a known sequence using unnatural amino acids, conformational restraints, isosteric replacement, and the like. The subject peptidomimetics constitute the continuum of structural space between peptides and nonpeptide synthetic structures; peptidomimetics may be useful, therefore, in delineating pharmacophores and in helping to translate peptides into nonpeptide compounds with the activity of the parent peptides.

Moreover, as is apparent from the present disclosure, mimotopes of the subject peptides can be provided. Such peptidomimetics can have such attributes as being non-hydrolysable (e.g., increased stability against proteases or other physiological conditions which degrade the corresponding peptide), increased specificity and/or potency, and increased cell permeability for intracellular localization of the peptidomimetic. For illustrative purposes, peptide analogs of the present invention can be generated using, for example, benzodiazepines (e.g., see Freidinger et al. in Peptides: Chemistry and Biology, G. R. Marshall ed., ESCOM Publisher: Leiden, Netherlands, 1988), substituted gama lactam rings (Garvey et al. in Peptides: Chemistry and Biology, G. R. Marshall ed., ESCOM Publisher: Leiden, Netherlands, 1988, p123), C-7 mimics (Huffman et al. in Peptides: Chemistry and Biology, G. R. Marshall ed., ESCOM Publisher: Leiden, Netherlands, 1988, p. 105), keto-methylene pseudopeptides (Ewenson et al. (1986) J Med Chem 29:295; and Ewenson et al. in Peptides: Structure and Function (Proceedings of the 9th American Peptide Symposium) Pierce Chemical Co. Rockland, Ill., 1985), β-turn dipeptide cores (Nagai et al. (1985) Tetrahedron Lett 26:647; and Sato et al. (1986) J Chem Soc Perkin Trans 1:1231), β-aminoalcohols (Gordon et al. (1985) Biochem Biophys Res Commun 126:419; and Dann et al. (1986) Biochem Biophys Res Commun 134:71), diaminoketones (Natarajan et al. (1984) Biochem Biophys Res Commun 124:141), and methyleneamino-modified (Roark et al. in Peptides: Chemistry and Biology, G. R. Marshall ed., ESCOM Publisher: Leiden, Netherlands, 1988, p134). Also, see generally, Session III: Analytic and synthetic methods, in in Peptides: Chemistry and Biology, G. R. Marshall ed., ESCOM Publisher: Leiden, Netherlands, 1988)

In addition to a variety of side chain replacements which can be carried out to generate the peptidomimetics, the present invention specifically contemplates the use of conformationally restrained mimics of peptide secondary structure. Numerous surrogates have been developed for the amide bond of peptides. Frequently exploited surrogates for the amide bond include the following groups (i) trans-olefins, (ii) fluoroalkene, (iii) methylencamino, (iv) phosphonamides, and (v) sulfonamides.

Moreover, other examples of mimetopes include, but are not limited to, protein-based compounds, carbohydrate-based compounds, lipid-based compounds, nucleic acid-based compounds, natural organic compounds, synthetically derived organic compounds, anti-idiotypic antibodies and/or catalytic antibodies, or fragments thereof. A mimetope can be obtained by, for example, screening libraries of natural and synthetic compounds for compounds capable of binding to the peptide of the invention. A mimetope can also be obtained, for example, from libraries of natural and synthetic compounds, in particular, chemical or combinatorial libraries (i.e., libraries of compounds that differ in sequence or size but that have the same building blocks). A mimetope can also be obtained by, for example, rational drug design. In a rational drug design procedure, the three-dimensional structure of a compound of the present invention can be analyzed by, for example, nuclear magnetic resonance (NMR) or x-ray crystallography. The three-dimensional structure can then be used to predict structures of potential mimetopes by, for example, computer modelling, the predicted mimetope structures can then be produced by, for example, chemical synthesis, recombinant DNA technology, or by isolating a mimetope from a natural source (e.g., plants, animals, bacteria and fungi).

A peptide of the invention may be synthesized by conventional techniques. For example, the peptides may be synthesized by chemical synthesis using solid phase peptide synthesis. These methods employ either solid or solution phase synthesis methods (see for example, J. M. Stewart, and J. D. Young, Solid Phase Peptide Synthesis, 2^{nd} Ed., Pierce Chemical Co., Rockford Ill. (1984) and G. Barany and R. B. Merrifield, The Peptides: Analysis Synthesis, Biology editors E. Gross and J. Meienhofer Vol. 2 Academic Press, New York, 1980, pp. 3-254 for solid phase synthesis techniques; and M Bodansky, Principles of Peptide Synthesis, Springer-Verlag, Berlin 1984, and E. Gross and J. Meienhofer, Eds., The Peptides: Analysis, Synthesis, Biology, suprs, Vol 1, for classical solution synthesis.) By way of example, a peptide may be synthesized using 9-fluorenyl methoxycarbonyl (Fmoc) solid phase chemistry with direct incorporation of phosphothreonine as the N-fluorenylmethoxy-carbonyl-O-benzyl-L-phosphothreonine derivative.

N-terminal or C-terminal fusion proteins comprising a peptide of the invention conjugated with other molecules may be prepared by fusing, through recombinant techniques, the N-terminal or C-terminal of the peptide, and the sequence of a selected protein or selectable marker with a desired biological function. The resultant fusion proteins contain the cytokine or recombinase fused to the selected protein or marker protein as described herein. Examples of proteins which may be used to prepare fusion proteins include immunoglobulins, glutathione-S-transferase (GST), hemagglutinin (HA), and truncated myc.

Peptides of the invention may be developed using a biological expression system. The use of these systems allows the production of large libraries of random peptide sequences and the screening of these libraries for peptide sequences that bind to particular proteins. Libraries may be produced by cloning synthetic DNA that encodes random peptide sequences into appropriate expression vectors. (See Christian et al 1992, J. Mol. Biol. 227:711; Devlin et al, 1990 Science 249:404; Cwirla et al 1990, Proc. Natl. Acad, Sci. USA, 87:6378). Libraries may also be constructed by concurrent synthesis of overlapping peptides (see U.S. Pat. No. 4,708,871).

The peptides of the invention may be converted into pharmaceutical salts by reacting with inorganic acids such as hydrochloric acid, sulfuric acid, hydrobromic acid, phosphoric acid, etc., or organic acids such as formic acid, acetic acid, propionic acid, glycolic acid, lactic acid, pyruvic acid, oxalic acid, succinic acid, malic acid, tartaric acid, citric acid, benzoic acid, salicylic acid, benezenesulfonic acid, and toluenesulfonic acids.

### Nucleic Acids

In certain aspects, one or more of the compositions described herein are isolated nucleic acid molecules. For example, in certain embodiments, the cytokine composition comprises an isolated nucleic acid molecule encoding one or more cytokines. In one embodiment, the antigen receptor composition comprises an isolated nucleic acid molecule encoding one or more TCR or CAR. In one embodiment, the integration composition comprises an isolated nucleic acid molecule encoding a recombinase or integrase. For example, in one embodiment, the one or more isolated nucleic acid molecule encodes one or more of peptides or proteins described herein, including, but not limited to, CCL2, CCL3, CCL4, CCL5, MIP-1α, CXCL9, CXCL10, CXCL12, CXCL16, CC17, CCL19, CCL20, CCL21, CCL22, or CCL27, a TCR, a CAR, a tyrosine recombinase, serine recombinase, bacteriophage integrase, tyrosine integrase, serine integrase, ΦC31 integrase, Cre recombinase, Flp recombinase, Bxb1 integrase, retroviral integrase, or a fragment or a variant thereof.

In various embodiments, the isolated nucleic acids include both DNA and RNA molecules. For example, in one embodiment, the method includes administration of an RNA molecule encoding an antigen receptor and a retroviral integrase for integration of the RNA molecule into a T cell. In another embodiment, the method includes administration of a DNA molecule encoding an antigen receptor and a bacteripohage integrase for integration of the DNA molecule into a T cell.

Further, the invention encompasses an isolated nucleic acid comprising a nucleotide sequence having substantial homology to a nucleotide sequence encoding one or more of peptides or proteins as disclosed herein. The nucleic acid sequence which is “substantially homologous” is at least about 50% identical, at least about 70% identical, at least about 80% identical, at least about 85% identical, at least about 86 identical, at least about 87% identical, at least about 88% identical, at least about 89% identical, at least about 90% identical, at least about 91% identical, at least about 92% identical, at least about 93% identical, at least about 94% identical, at least about 95% identical, at least about 96% identical, at least about 97% identical, at least about 98% identical, at least about 99% identical, to a nucleotide sequence of an isolated nucleic acid encoding a peptide of the invention.

Thus, the invention encompasses expression vectors and methods for the introduction of exogenous nucleic acid molecules into cells with concomitant expression of the exogenous nucleic acid molecules in the cells such as those described, for example, in Sambrook et al. (2012, Molecular Cloning: A Laboratory Manual, Cold Spring Harbor Laboratory, New York), and in Ausubel et al. (1997, Current Protocols in Molecular Biology, John Wiley & Sons, New York).

The desired nucleic acid encoding one or more of the peptides or proteins described herein can be cloned into a number of types of vectors. However, the present invention should not be construed to be limited to any particular vector. Instead, the present invention should be construed to encompass a wide plethora of vectors which are readily available and/or well-known in the art. For example, a desired polynucleotide of the invention can be cloned into a vector including, but not limited to a plasmid, a phagemid, a phage derivative, an animal virus, and a cosmid. Vectors of particular interest include expression vectors, replication vectors, probe generation vectors, and sequencing vectors.

In specific embodiments, the expression vector is selected from the group consisting of a viral vector, a bacterial vector and a mammalian cell vector. Numerous expression vector systems exist that comprise at least a part or all of the compositions discussed above. Prokaryote- and/or eukaryote-vector based systems can be employed for use with the present invention to produce polynucleotides, or their cognate polypeptides. Many such systems are commercially and widely available.

Further, the expression vector may be provided to a cell in the form of a viral vector. Viral vector technology is well known in the art and is described, for example, in Sambrook et al. (2012), and in Ausubel et al. (1997), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. (See, e.g., WO 01/96584; WO 01/29058; and U.S. Pat. No. 6,326,193.

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

For example, vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. In one embodiment, the composition includes a vector derived from an adeno-associated virus (AAV). Adeno-associated viral (AAV) vectors have become powerful gene delivery tools for the treatment of various disorders. AAV vectors possess a number of features that render them ideally suited for gene therapy, including a lack of pathogenicity, minimal immunogenicity, and the ability to transduce postmitotic cells in a stable and efficient manner. Expression of a particular gene contained within an AAV vector can be specifically targeted to one or more types of cells by choosing the appropriate combination of AAV serotype, promoter, and delivery method

In one embodiment, the encoding sequence is contained within an AAV vector. More than 30 naturally occurring serotypes of AAV are available. Many natural variants in the AAV capsid exist, allowing identification and use of an AAV with properties specifically suited for skeletal muscle. AAV viruses may be engineered using conventional molecular biology techniques, making it possible to optimize these particles for cell specific delivery of nucleic acid sequences, for minimizing immunogenicity, for tuning stability and particle lifetime, for efficient degradation, for accurate delivery to the nucleus, etc.

Thus, expression of one or more proteins can be achieved by delivering a recombinantly engineered AAV or artificial AAV that contains one or more encoding sequences. The use of AAVs is a common mode of exogenous delivery of DNA as it is relatively non-toxic, provides efficient gene transfer, and can be easily optimized for specific purposes. Exemplary AAV serotypes include, but is not limited to AAV1, AAV2, AAV3, AAV4, AAV5, AAV6, AAV7, AAV8 and AAV9.

Desirable AAV fragments for assembly into vectors include the cap proteins, including the vp1, vp2, vp3 and hypervariable regions, the rep proteins, including rep 78, rep 68, rep 52, and rep 40, and the sequences encoding these proteins. These fragments may be readily utilized in a variety of vector systems and host cells. Such fragments may be used alone, in combination with other AAV serotype sequences or fragments, or in combination with elements from other AAV or non-AAV viral sequences. As used herein, artificial AAV serotypes include, without limitation, AAV with a non-naturally occurring capsid protein. Such an artificial capsid may be generated by any suitable technique, using a selected AAV sequence (e.g., a fragment of a vp1 capsid protein) in combination with heterologous sequences which may be obtained from a different selected AAV serotype, non-contiguous portions of the same AAV serotype, from a non-AAV viral source, or from a non-viral source. An artificial AAV serotype may be, without limitation, a chimeric AAV capsid, a recombinant AAV capsid, or a “humanized” AAV capsid. Thus exemplary AAVs, or artificial AAVs, suitable for expression of one or more proteins, include AAV2/8 (see U.S. Pat. No. 7,282,199), AAV2/5 (available from the National Institutes of Health), AAV2/9 (International Patent Publication No. WO2005/033321), AAV2/6 (U.S. Pat. No. 6,156,303), and AAVrh8 (International Patent Publication No. WO2003/042397), among others.

For expression of the desired polynucleotide, at least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 genes, a discrete element overlying the start site itself helps to fix the place of initiation.

Additional promoter elements, i.e., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either co-operatively or independently to activate transcription.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (U.S. Pat. Nos. 4,683,202, 5,928,906). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression, for example, see Sambrook et al. (2012). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

In order to assess the expression of the desired polynucleotide, the expression vector to be introduced into a cell can also contain either a selectable marker gene or a reporter gene or both to facilitate identification and selection of expressing cells from the population of cells sought to be transfected or infected through viral vectors. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neo and the like.

Reporter genes are used for identifying potentially transfected cells and for evaluating the functionality of regulatory sequences. Reporter genes that encode for easily assayable proteins are well known in the art. In general, a reporter gene is a gene that is not present in or expressed by the recipient organism or tissue and that encodes a protein whose expression is manifested by some easily detectable property, e.g., enzymatic activity. Expression of the reporter gene is assayed at a suitable time after the DNA has been introduced into the recipient cells.

Suitable reporter genes may include genes encoding luciferase, beta-galactosidase, chloramphenicol acetyl transferase, secreted alkaline phosphatase, or the green fluorescent protein gene (see, e.g., Ui-Tei et al., 2000 FEBS Lett. 479:79-82). Suitable expression systems are well known and may be prepared using well known techniques or obtained commercially. Internal deletion constructs may be generated using unique internal restriction sites or by partial digestion of non-unique restriction sites. Constructs may then be transfected into cells that display high levels of siRNA polynucleotide and/or polypeptide expression. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In the context of an expression vector, the vector can be readily introduced into a host cell, e.g., mammalian, bacterial, yeast or insect cell by any method in the art. For example, the expression vector can be transferred into a host cell by physical, chemical or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2012, Molecular Cloning: A Laboratory Manual, Cold Spring Harbor Laboratory, New York), and in Ausubel et al. (1997, Current Protocols in Molecular Biology, John Wiley & Sons, New York).

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (i.e., an artificial membrane vesicle). The preparation and use of such systems is well known in the art.

Regardless of the method used to introduce exogenous nucleic acids into a host cell, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

Any DNA vector or delivery vehicle can be utilized to transfer the desired polynucleotide to a cell in vitro or in vivo. In the case where a non-viral delivery system is utilized, an exemplary delivery vehicle is a liposome. The above-mentioned delivery systems and protocols therefore can be found in Gene Targeting Protocols, 2ed., pp 1-35 (2002) and Gene Transfer and Expression Protocols, Vol. 7, Murray ed., pp 81-89 (1991).

“Liposome” is a generic term encompassing a variety of single and multilamellar lipid vehicles formed by the generation of enclosed lipid bilayers or aggregates. Liposomes may be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers. However, the present invention also encompasses compositions that have different structures in solution than the normal vesicular structure. For example, the lipids may assume a micellar structure or merely exist as nonuniform aggregates of lipid molecules. Also contemplated are lipofectamine-nucleic acid complexes.

In one embodiment, the composition of the invention comprises in vitro transcribed (IVT) RNA encoding one or more components of the one or more proteins. In one embodiment, an IVT RNA can be introduced to a cell as a form of transient transfection. The RNA is produced by in vitro transcription using a plasmid DNA template generated synthetically. DNA of interest from any source can be directly converted by PCR into a template for in vitro mRNA synthesis using appropriate primers and RNA polymerase. The source of the DNA can be, for example, genomic DNA, plasmid DNA, phage DNA, cDNA, synthetic DNA sequence or any other appropriate source of DNA. The desired template for in vitro transcription is one or more proteins or protein fragment.

In one embodiment, the DNA to be used for PCR contains an open reading frame. The DNA can be from a naturally occurring DNA sequence from the genome of an organism. In one embodiment, the DNA is a full length gene of interest of a portion of a gene. The gene can include some or all of the 5′ and/or 3′ untranslated regions (UTRs). The gene can include exons and introns. In one embodiment, the DNA to be used for PCR is a human gene. In another embodiment, the DNA to be used for PCR is a human gene including the 5′ and 3′ UTRs. The DNA can alternatively be an artificial DNA sequence that is not normally expressed in a naturally occurring organism. An exemplary artificial DNA sequence is one that contains portions of genes that are ligated together to form an open reading frame that encodes a fusion protein. The portions of DNA that are ligated together can be from a single organism or from more than one organism.

In one embodiment, the composition of the present invention comprises a modified nucleic acid encoding one or more proteins described herein. For example, in one embodiment, the composition comprises a nucleoside-modified RNA. In one embodiment, the composition comprises a nucleoside-modified mRNA. Nucleoside-modified mRNA have particular advantages over non-modified mRNA, including for example, increased stability, low immunogenicity, and enhanced translation. Nucleoside-modified mRNA useful in the present invention is further described in U.S. Pat. No. 8,278,036, which is incorporated by reference herein in its entirety.

### Therapeutic Methods

In one aspect, the present invention provides methods to treat a disease or disorder in a subject in need thereof. In one embodiment, the method of the present invention comprises administering to a subject, a combination of a cytokine composition, an antigen receptor composition, and an integration composition, as described herein.

The method of the present invention is used to treat any type of disease or disorder associated with the antigen that is recognized by the antigen receptor encoded by the antigen receptor composition, including, but not limited to cancer and pathogenic diseases and disorders.

Pathogenic diseases and disorders that can be treated by the disclosed methods include, but are not limited to, bacterial infection, viral infections, fungal infections, and diseases or disorders associated with a parasite.

The following are non-limiting examples of cancers that can be treated by the disclosed methods: acute lymphoblastic leukemia, acute myeloid leukemia, adrenocortical carcinoma, appendix cancer, basal cell carcinoma, bile duct cancer, bladder cancer, bone cancer, brain and spinal cord tumors, brain stem glioma, brain tumor, breast cancer, bronchial tumors, burkitt lymphoma, carcinoid tumor, central nervous system atypical teratoid/rhabdoid tumor, central nervous system embryonal tumors, central nervous system lymphoma, cerebellar astrocytoma, cerebral astrocytoma/malignant glioma, cerebral astrocytotna/malignant glioma, cervical cancer, childhood visual pathway tumor, chordoma, chronic lymphocytic leukemia, chronic myelogenous leukemia, chronic myeloproliferative disorders, colon cancer, colorectal cancer, craniopharyngioma, cutaneous cancer, cutaneous t-cell lymphoma, endometrial cancer, ependymoblastoma, ependymoma, esophageal cancer, ewing family of tumors, extracranial cancer, extragonadal germ cell tumor, extrahepatic bile duct cancer, extrahepatic cancer, eye cancer, fungoides, gallbladder cancer, gastric (stomach) cancer, gastrointestinal cancer, gastrointestinal carcinoid tumor, gastrointestinal stromal tumor (gist), germ cell tumor, gestational cancer, gestational trophoblastic tumor, glioblastoma, glioma, hairy cell leukemia, head and neck cancer, hepatocellular (liver) cancer, histiocytosis, hodgkin lymphoma, hypopharyngeal cancer, hypothalamic and visual pathway glioma, hypothalamic tumor, intraocular (eye) cancer, intraocular melanoma, islet cell tumors, kaposi sarcoma, kidney (renal cell) cancer, langerhans cell cancer, langerhans cell histiocytosis, laryngeal cancer, leukemia, lip and oral cavity cancer, liver cancer, lung cancer, lymphoma, macroglobulinemia, malignant fibrous histiocvtoma of bone and osteosarcoma, medulloblastoma, medulloepithelioma, melanoma, merkel cell carcinoma, mesothelioma, metastatic squamous neck cancer with occult primary, mouth cancer, multiple endocrine neoplasia syndrome, multiple myeloma, mycosis, myelodysplastic syndromes, myelodysplastic/myeloproliferative diseases, myelogenous leukemia, myeloid leukemia, myeloma, myeloproliferative disorders, nasal cavity and paranasal sinus cancer, nasopharyngeal cancer, neuroblastoma, non-hodgkin lymphoma, non-small cell lung cancer, oral cancer, oral cavity cancer, oropharyngeal cancer, osteosarcoma and malignant fibrous histiocytoma, osteosarcoma and malignant fibrous histiocytoma of bone, ovarian, ovarian cancer, ovarian epithelial cancer, ovarian germ cell tumor, ovarian low malignant potential tumor, pancreatic cancer, papillomatosis, paraganglioma, parathyroid cancer, penile cancer, pharyngeal cancer, pheochromocytoma, pineal parenchymal tumors of intermediate differentiation, pincoblastoma and supratentorial primitive neuroectodermal tumors, pituitary tumor, plasma cell neoplasm, plasma cell neoplasm/multiple myeloma, pleuropulmonary blastoma, primary central nervous system cancer, primary central nervous system lymphoma, prostate cancer, rectal cancer, renal cell (kidney) cancer, renal pelvis and ureter cancer, respiratory tract carcinoma involving the nut gene on chromosome 15, retinoblastoma, rhabdomyosarcoma, salivary gland cancer, sarcoma, sezary syndrome, skin cancer (melanoma), skin cancer (nonmelanoma), skin carcinoma, small cell lung cancer, small intestine cancer, soft tissue cancer, soft tissue sarcoma, squamous cell carcinoma, squamous neck cancer, stomach (gastric) cancer, supratentorial primitive neuroectodermal tumors, supratentorial primitive neuroectodermal tumors and pincoblastoma, T-cell lymphoma, testicular cancer, throat cancer, thymoma and thymic carcinoma, thyroid cancer, transitional cell cancer, transitional cell cancer of the renal pelvis and ureter, trophoblastic tumor, urethral cancer, uterine cancer, uterine sarcoma, vaginal cancer, visual pathway and hypothalamic glioma, vulvar cancer, waldenstrom macroglobulinemia, and wilms tumor.

Compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials. When “an effective amount”, or “therapeutic amount” is indicated, the precise amount of the compositions of the present invention to be administered can be determined by a physician with consideration of individual differences in age, weight, disease progression, and condition of the patient (subject). The optimal dosage and treatment regime for a particular patient can readily be determined by one skilled in the art of medicine by monitoring the subject for signs of disease and adjusting the treatment accordingly.

The administration of the subject compositions may be carried out in any convenient manner, including by aerosol inhalation, injection, ingestion, transfusion, implantation or transplantation. The compositions described herein may be administered to a subject subcutaneously, intradermally, intratumorally, intranodally, intramedullary, intramuscularly, by intravenous (i.v.) injection, or intraperitoneally.

In one embodiment, the method of the invention comprises a local administration of a cytokine composition and subsequent local administration of an antigen receptor composition. In one embodiment, the method comprises a local administration of a cytokine composition and subsequent local administration of an antigen receptor composition at the same site as where the cytokine composition was administered.

In one embodiment, the antigen receptor composition is administered one or more days, 2 or more days, 3 or more days, 4 or more days, 5 or more days, 6 or more days, 7 or more days, 8 or more days, 9 or more days, 10 or more days, 11 or more days, 12 or more days, 13 or more days, or 14 or more days, after the cytokine composition is administered. In one embodiment, the antigen receptor composition is administered one or more weeks, 2 or more weeks, 3 or more weeks, 4 or more weeks, 5 or more weeks, 6 or more weeks, 7 or more weeks, 8 or more weeks, 9 or more weeks, 10 or more weeks, 11 or more weeks, or 12 or more weeks, after the cytokine composition is administered. In one embodiment, the antigen receptor composition is administered one or more months, 2 or more months, 3 or more months, 4 or more months, 5 or more months, 6 or more months, 7 or more months, 8 or more months, 9 or more months, 10 or more months, 11 or more months, or 12 or more months, after the cytokine composition is administered.

In certain embodiments, the method comprises administering the integration composition to the subject to enable integration of the nucleic acid sequence encoding the antigen receptor into the DNA of a T cell of the subject. In certain embodiments, the integration composition is administered at the same site as where the antigen receptor composition is administered.

In certain embodiments, the integration composition is administered at the same time as when the antigen receptor composition is administered. In certain embodiments, the integration composition is administered after the antigen receptor composition is administered. In certain embodiments, the integration composition is administered before the antigen receptor composition is administered.

In certain embodiments, the method comprises repeated administration of one or more of the compositions. For example, in one embodiment, the method comprises administering a cytokine composition; administering an antigen receptor composition either with or without accompanying co-administration of an integration composition; and administering an antigen receptor composition either without accompanying co-administration of an integration composition at least one more time. In one embodiment, the method comprises administering a cytokine composition; administering an antigen receptor composition either with or without accompanying co-administration of an integration composition; administering a cytokine composition at least one more time; and administering an antigen receptor composition either without accompanying co-administration of an integration composition at least one more time.

Forms of administration that may be useful in the methods described herein include, but are not limited to, direct delivery to a desired organ, oral, inhalation, intranasal, intratracheal, intravenous, intramuscular, intratumroal, subcutaneous, intradermal, and other parental routes of administration. Additionally, routes of administration may be combined, if desired. In one embodiments, route of administration is intradermal injection or intratumoral injection. In one embodiment, one or more composition is administered to a treatment site during a surgical procedure, for example during surgical resection of all or part of a tumor.

In certain embodiments of the present invention, the composition, as described herein, are administered to a subject in conjunction with (e.g. before, simultaneously, or following) any number of relevant treatment modalities including but not limited to treatment with agents such as antiviral therapy, cidofovir and interleukin-2, Cytarabine (also known as ARA-C) or natalizumab treatment for MS patients or efalizumab treatment for psoriasis patients or other treatments for PML patients. In further embodiments, the compositions of the invention may be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycophenolic acid, steroids, FR901228, cytokines, and irradiation. These drugs inhibit either the calcium dependent phosphatase calcineurin (cyclosporine and FK506) or inhibit the p70S6 kinase that is important for growth factor induced signaling (rapamycin) (Liu et al., Cell 66:807-815, 1991; Henderson et al., Immun. 73:316-321, 1991; Bierer et al., Curr. Opin. Immun. 5:763-773, 1993).

In certain embodiments, one or more of the compositions are administered to the subject in vivo, to allow for direct genetic engineering of the subject's T cells without the need for ex vivo manipulation. In vivo delivery of the composition can be carried out using any known delivery technique or strategy. For example, in vivo delivery of a nucleic acid molecule described herein can be carried out using electroporation, laser or light-mediated photoporation, microinjection, and liposome- or polymer-based nanocarriers.

### Dosage and Formulation (Compositions)

The present invention envisions treating a disease, for example, cancer or diseases associated with a pathogen, in a subject by the administration of one or more of the therapeutic agents of the present invention (e.g., the cytokine composition, antigen receptor composition and integration composition).

Administration of the composition in accordance with the present invention may be continuous or intermittent, depending, for example, upon the recipient's physiological condition, whether the purpose of the administration is therapeutic or prophylactic, and other factors known to skilled practitioners. The administration of the agents of the invention may be essentially continuous over a preselected period of time or may be in a series of spaced doses. In one embodiment, the cytokine composition, the antigen receptor composition, and the integration composition of the invention are administered locally to the same site. The amount administered will vary depending on various factors including, but not limited to, the composition chosen, the particular disease, the weight, the physical condition, and the age of the mammal, and whether prevention or treatment is to be achieved. Such factors can be readily determined by the clinician employing animal models or other test systems which are well known to the art.

One or more suitable unit dosage forms having the therapeutic agent(s) of the invention, which, as discussed below, may optionally be formulated for sustained release (for example using microencapsulation, see WO 94/07529, and U.S. Pat. No. 4,962,091 the disclosures of which are incorporated by reference herein), can be administered by a variety of routes including parenteral, including by intravenous and intramuscular routes, as well as by direct injection into the diseased tissue. For example, the therapeutic agent may be directly injected into a tumor. The formulations may, where appropriate, be conveniently presented in discrete unit dosage forms and may be prepared by any of the methods well known to pharmacy. Such methods may include the step of bringing into association the therapeutic agent with liquid carriers, solid matrices, semi-solid carriers, finely divided solid carriers or combinations thereof, and then, if necessary, introducing or shaping the product into the desired delivery system.

In certain embodiments, the therapeutic agent is combined with a pharmaceutically acceptable carrier, diluent or excipient to form a pharmaceutical formulation, or unit dosage form. The total active ingredients in such formulations include from 0.1 to 99.9% by weight of the formulation. A “pharmaceutically acceptable” is a carrier, diluent, excipient, and/or salt that is compatible with the other ingredients of the formulation, and not deleterious to the recipient thereof. The active ingredient for administration may be present as a powder or as granules; as a solution, a suspension or an emulsion.

Pharmaceutical formulations containing the therapeutic agents of the invention can be prepared by procedures known in the art using well known and readily available ingredients. The therapeutic agents of the invention can also be formulated as solutions appropriate for parenteral administration, for instance by intramuscular, subcutaneous or intravenous routes.

The pharmaceutical formulations of the therapeutic agents of the invention can also take the form of an aqueous or anhydrous solution or dispersion, or alternatively the form of an emulsion or suspension.

Thus, the therapeutic agent may be formulated for parenteral administration (e.g., by injection, for example, bolus injection or continuous infusion) and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

It will be appreciated that the unit content of active ingredient or ingredients contained in an individual aerosol dose of each dosage form need not in itself constitute an effective amount for treating the particular indication or disease since the necessary effective amount can be reached by administration of a plurality of dosage units. Moreover, the effective amount may be achieved using less than the dose in the dosage form, either individually, or in a series of administrations.

The pharmaceutical formulations of the present invention may include, as optional ingredients, pharmaceutically acceptable carriers, diluents, solubilizing or emulsifying agents, and salts of the type that are well-known in the art. Specific non-limiting examples of the carriers and/or diluents that are useful in the pharmaceutical formulations of the present invention include water and physiologically acceptable buffered saline solutions, such as phosphate buffered saline solutions pH 7.0-8.0.

The expression vectors, transduced cells, polynucleotides and polypeptides (active ingredients) of this invention can be formulated and administered to treat a variety of disease states by any means that produces contact of the active ingredient with the agent's site of action in the body of the organism. They can be administered by any conventional means available for use in conjunction with pharmaceuticals, either as individual therapeutic active ingredients or in a combination of therapeutic active ingredients. They can be administered alone, but are generally administered with a pharmaceutical carrier selected on the basis of the chosen route of administration and standard pharmaceutical practice.

In general, water, suitable oil, saline, aqueous dextrose (glucose), and related sugar solutions and glycols such as propylene glycol or polyethylene glycols are suitable carriers for parenteral solutions. Solutions for parenteral administration contain the active ingredient, suitable stabilizing agents and, if necessary, buffer substances. Antioxidizing agents such as sodium bisulfate, sodium sulfite or ascorbic acid, either alone or combined, are suitable stabilizing agents. Also used are citric acid and its salts and sodium Ethylenediaminetetraacetic acid (EDTA). In addition, parenteral solutions can contain preservatives such as benzalkonium chloride, methyl- or propyl-paraben and chlorobutanol. Suitable pharmaceutical carriers are described in Remington's Pharmaceutical Sciences, a standard reference text in this field.

The active ingredients of the invention may be formulated to be suspended in a pharmaceutically acceptable composition suitable for use in mammals and in particular, in humans. Such formulations include the use of adjuvants such as muramyl dipeptide derivatives (MDP) or analogs that are described in U.S. Pat. Nos. 4,082,735; 4,082,736; 4,101,536; 4,185,089; 4,235,771; and 4,406,890. Other adjuvants, which are useful, include alum (Pierce Chemical Co.), lipid A, trehalose dimycolate and dimethyldioctadecylammonium bromide (DDA), Freund's adjuvant, and IL-12. Other components may include a polyoxypropylene-polyoxyethylene block polymer (Pluronic®), a non-ionic surfactant, and a metabolizable oil such as squalene (U.S. Pat. No. 4,606,918).

Additionally, standard pharmaceutical methods can be employed to control the duration of action. These are well known in the art and include control release preparations and can include appropriate macromolecules, for example polymers, polyesters, polyamino acids, polyvinyl, pyrolidone, ethylenevinylacetate, methyl cellulose, carboxymethyl cellulose or protamine sulfate. The concentration of macromolecules as well as the methods of incorporation can be adjusted in order to control release. Additionally, the agent can be incorporated into particles of polymeric materials such as polyesters, polyamino acids, hydrogels, poly (lactic acid) or ethylenevinylacetate copolymers. In addition to being incorporated, these agents can also be used to trap the compound in microcapsules.

Accordingly, the composition of the present invention may be delivered via various routes and to various sites in a mammal body to achieve a particular effect (see, e.g., Rosenfeld et al., 1991; Rosenfeld et al., 1991a; Jaffe et al., supra; Berkner, supra). One skilled in the art will recognize that although more than one route can be used for administration, a particular route can provide a more immediate and more effective reaction than another route. In one embodiment, the composition described above is administered to the subject by intratumoral injection. Other forms of administration that may be useful in the methods described herein include, but are not limited to, direct delivery to a desired organ, intramuscular, subcutaneous, intradermal, and other parental routes of administration.

The active ingredients of the present invention can be provided in unit dosage form wherein each dosage unit, e.g., a teaspoonful, tablet, solution, or suppository, contains a predetermined amount of the composition, alone or in appropriate combination with other active agents. The term “unit dosage form” as used herein refers to physically discrete units suitable as unitary dosages for human and mammal subjects, each unit containing a predetermined quantity of the compositions of the present invention, alone or in combination with other active agents, calculated in an amount sufficient to produce the desired effect, in association with a pharmaceutically acceptable diluent, carrier, or vehicle, where appropriate. The specifications for the unit dosage forms of the present invention depend on the particular effect to be achieved and the particular pharmacodynamics associated with the composition in the particular host.

These methods described herein are by no means all-inclusive, and further methods to suit the specific application will be apparent to the ordinary skilled artisan. Moreover, the effective amount of the compositions can be further approximated through analogy to compounds known to exert the desired effect.

### Gene Therapy Administration

One skilled in the art recognizes that different methods of delivery may be utilized to administer a nucleic acid molecule (e.g., a vector) into a cell. Examples include: (1) methods utilizing physical means, such as electroporation (electricity), a gene gun (physical force) or applying large volumes of a liquid (pressure); and (2) methods wherein the vector is complexed to another entity, such as a liposome, aggregated protein or transporter molecule.

Furthermore, the actual dose and schedule can vary depending on whether the compositions are administered in combination with other compositions, or depending on interindividual differences in pharmacokinetics, drug disposition, and metabolism. Similarly, amounts can vary in in vitro applications depending on the particular cell line utilized (e.g., based on the number of vector receptors present on the cell surface, or the ability of the particular vector employed for gene transfer to replicate in that cell line). Furthermore, the amount of vector to be added per cell will likely vary with the length and stability of the therapeutic gene inserted in the vector, as well as also the nature of the sequence, and is particularly a parameter which needs to be determined empirically, and can be altered due to factors not inherent to the methods of the present invention (for instance, the cost associated with synthesis). One skilled in the art can easily make any necessary adjustments in accordance with the exigencies of the particular situation.

The nucleic acid molecule may also contain a suicide gene i.e., a gene which encodes a product that can be used to destroy the cell. In many gene therapy situations, it is desirable to be able to express a gene for therapeutic purposes in a host, cell but also to have the capacity to destroy the host cell at will. The therapeutic agent can be linked to a suicide gene, whose expression is not activated in the absence of an activator compound. When death of the cell in which both the agent and the suicide gene have been introduced is desired, the activator compound is administered to the cell thereby activating expression of the suicide gene and killing the cell. Examples of suicide gene/prodrug combinations which may be used are herpes simplex virus-thymidine kinase (HSV-tk) and ganciclovir, acyclovir; oxidoreductase and cycloheximide; cytosine deaminase and 5-fluorocytosine; thymidine kinase thymidilate kinase (Tdk::Tmk) and AZT; and deoxycytidine kinase and cytosine arabinoside.

### Kits

The invention also includes a kit comprising one or more of the compositions described herein. For example, in one embodiment, the kit comprises one or more of: a cytokine composition, an antigen receptor composition, and an integration composition, as described herein. In one embodiment, the kit comprises a cytokine composition, an antigen receptor composition, and an integration composition, as described herein. In one embodiment, the kit comprises instructional material which describes the use of the composition. For instance, in some embodiments, the instructional material describes administering the composition(s), to a subject as a therapeutic treatment or a non-treatment use as described elsewhere herein. In an embodiment, this kit further comprises a (optionally sterile) pharmaceutically acceptable carrier suitable for dissolving or suspending the composition(s), for instance, prior to administering the composition(s) to a subject. Optionally, the kit comprises an applicator for administering the composition(s).

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1

The experiments described herein were conducted to examine whether tumor-reactive T cells engineered to express recombinant tumor antigen-specific T cell receptors (TCR) or chimeric antigen receptors (CAR) can be generated directly in cancer patients intralesionally or intradermally using non-viral gene therapy approaches with reduced toxicities. It is expected that this treatment will lead to the generation of the pool of the T cells expressing tumor-specific receptor (TCR or CAR) that allows T cell-mediated recognition and killing of the cancer cells.

### Plasmid DNA Constructs:

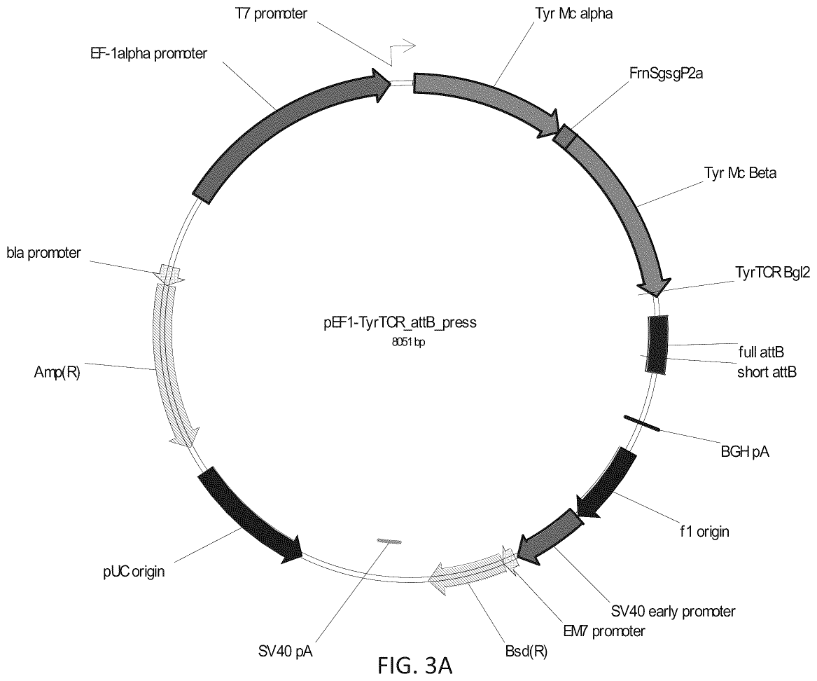

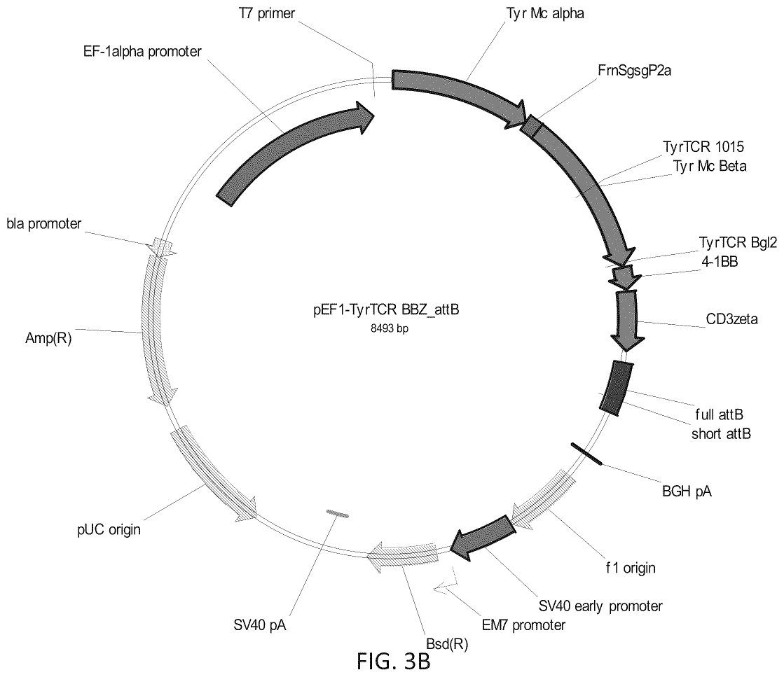

Human α/β tyrosinase-specific TCR (TyrTCR) sequence was amplified from pMSCV1 TyrAFBMc plasmid and inserted into pEF1-TOPO expression vector. Further, full-length attB sequence necessary for PhiC31 integrase-mediated genomic integration was amplified from pTA-attB plasmid and ligated into pEF1-Tyr-TCR plasmid (FIG. 3A).

To improve intracellular signaling from the recombinant TCR recombinant TCR B chain was fused with TCR-2, CD137 and CD28 signaling domains (as it was done in the design of the chimeric antigen receptors (CAR). It is expected that these signaling domains will enhance T cell effector function and permit more effective tumor targeting. This construct was designated as Tyr-TCR-BB-Zeta (FIG. 3B).

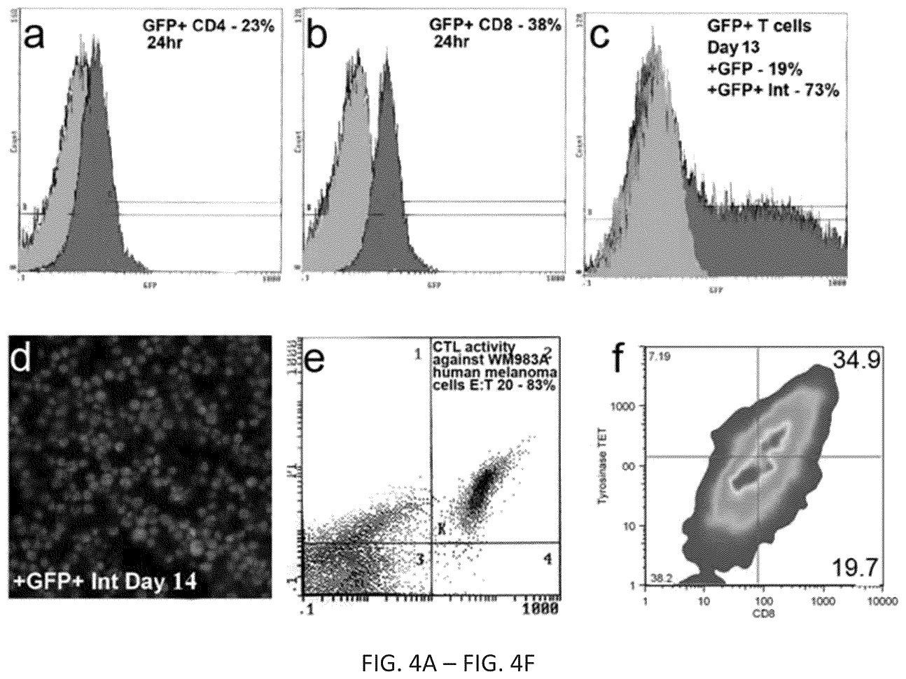

To assess the capacity of the non-viral ΦC31 integrase-mediated gene transfer, freshly isolated pan-T cells were co-transduced with GFP-attB and ΦC31-integrase encoding plasmids using Amaxa nucleofection reaction (electroporation of plasmid DNA). Nucleofection of the Pan T cells resulted in the expression of the transgene (GFP) in 23% and 38% of CD4+ and CD8+ T cells, respectively (FIG. 4A and FIG. 4B).

PhiC31 Integration of Plasmid DNA into Quiescent T Cells.

To assess the capacity of the non-viral ΦC31 integrase-mediated gene transfer, freshly isolated pan-T cells were co-transduced with EGFP-attB and ΦC31-integrase encoding plasmids. Nucleofection of the Pan T cells resulted in the expression of the transgene (EGFP) in 23% and 38% of CD4+ and CD8+ T cells, respectively (FIG. 4A and FIG. 4B). Stimulation of T cells with anti-CD3/CD28 antibodies in the presence of IL-2 for 2 weeks led to 40-fold expansion of the T cells with more than 70% of them expressing EGFP (FIG. 4C and FIG. 4D). Human T cells transduced with Tyr-TCR under these conditions showed high CTL activity against tyrosinase+ HLA-A2+ melanoma in vitro (FIG. 4E) with ˜35% of cultured CD8+ T cells showing binding to tyrosinase 368-376 tetramers (FIG. 4F).

When compared to prior studies (Frankel et al., J Immunol 2010, 184:5988-5998), ΦC31-mediated integration produced 2 times more recombinant Tyr-TCR+CD8+ T cells than γ-retroviral gene transfer, demonstrating that ΦC31-integrase-mediated gene transfer provides durable Tyr-TCR expression and production of cytotoxic T cells.

### Targeting of Melanoma Lesions In Vivo

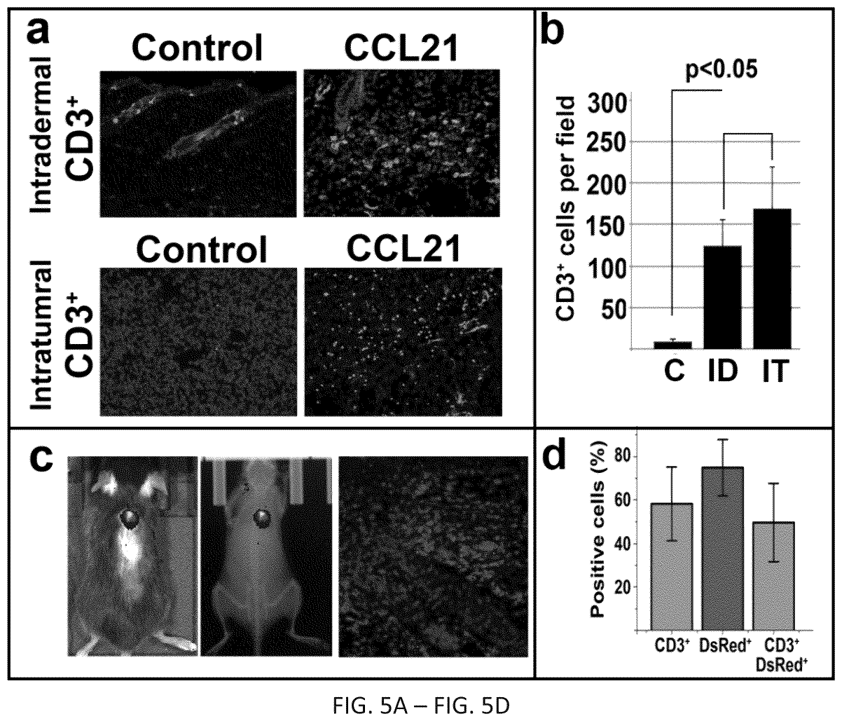

To obtain a proof-of-concept data, it was tested whether pre-conditioning of the skin or the tumor lesions with cytokines (CCL21 secondary lymphoid chemokine) leads to the infiltration of tissues with T cells. When plasmid DNA encoding CCL21 was in vivo electro orated into the skin or the established intradermal melanoma lesions, a significant infiltration of tissues with chemokines was observed. Infiltration is illustrated in FIG. 5A (indirect immunofluorescent detection of T cells). Quantitation is provided in FIG. 5B). To assess whether recombinant TCR could be delivered into chemokine-recruited T cells, 48 hours after pre-conditioning of the tissue with CCL21, a plasmid DNA encoding TyrTCR transcriptionally linked to DsRed fluorescent reporter was administered. As depicted in FIG. 5C, single in vivo electroporation of the TyrTCR reporter plasmid led to an expression of the construct in mouse skin, as visualized by the in vivo live animal imaging (FIG. 5C). Quantitation of DsRed+ T cells showed that about 50% of T cells extracted from the area expressed DsRed construct (FIG. 5D). Collectively, these studies demonstrated that Tyr-TCR+ T cells could be generated in vivo via electroporation of plasmid DNA.

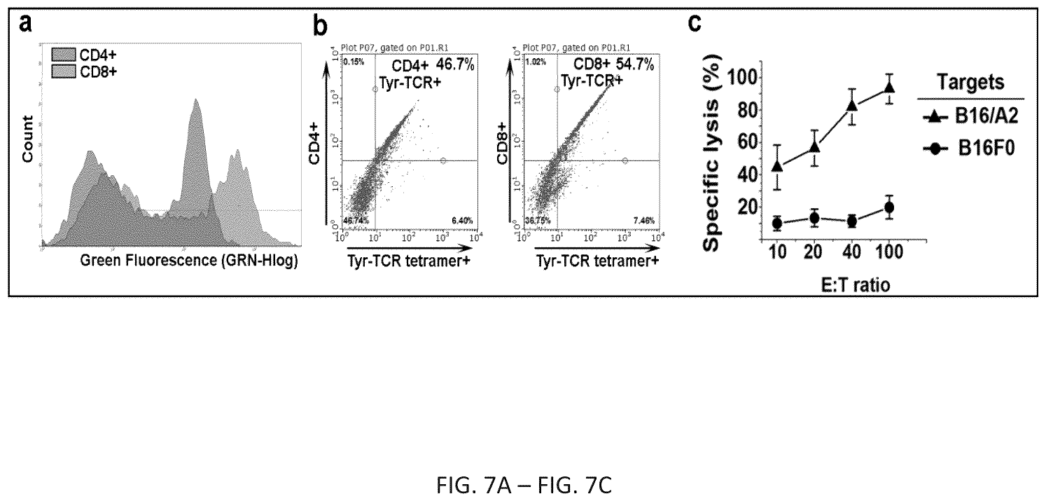

To determine whether in vivo gene transfer is suitable for tumor targeting in vivo, additional cohorts of B16/A2-bearing mice were treated with a mixture of Tyr-TCR and Tyr-TCR-BB-Zeta construct (10 μg per treatment) and with PhiC31-encoding plasmid (20 μg per treatment) intratumorally after priming of lesions with CCL21. In 48 hours melanoma lesions were excised from one experimental cohort and intratumoral T cells were extracted. FACS-based profiling demonstrated that the total T cells population was comprised of both CD4+ and CD8+ T cells (FIG. 7A). Dot plots showed that about 50% of CD4+ and CD8+ T cells also expressed recombinant TCR as detected by binding of fluorescently labeled TyrTCR-specific tetramer to the cells (FIG. 7B). These T cells also showed a substantial cytotoxic activity against tyrosinase-positive HLA-A2 positive melanoma cells in vitro at different Effector:Taget (E:T) ratios (FIG. 7C).

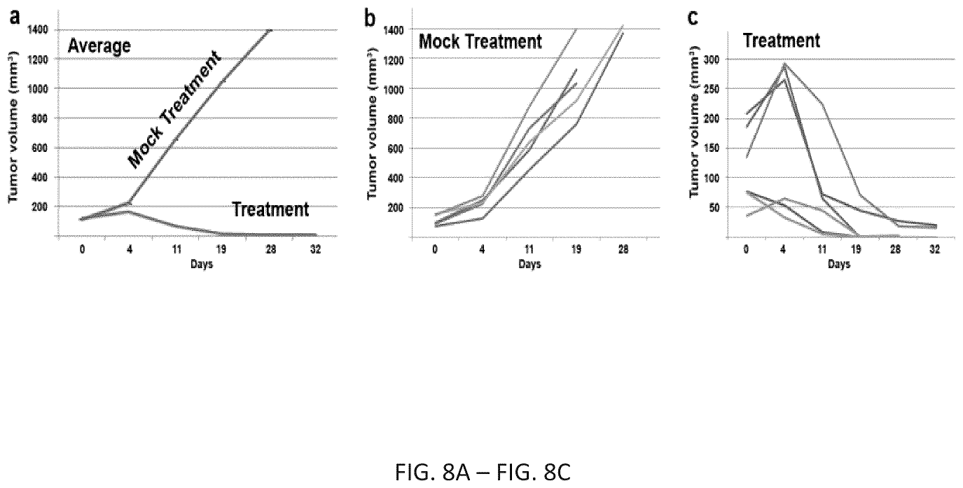

Remaining animals were further treated with CCL21-preconditioning and Tyr-TCR-PhiC31 in vivo electroporation for 3 more times for a total of 4 consecutive intratumoral treatments. Within 4 weeks, 4 consecutive treatments of the established lesions led to a complete (70% of animals) or partial (30% of animals) remission of the intradermal melanomas (FIG. 8A and FIG. 8C), whereas control, mock treated lesions continue to progress (FIG. 8A and FIG. 8B).

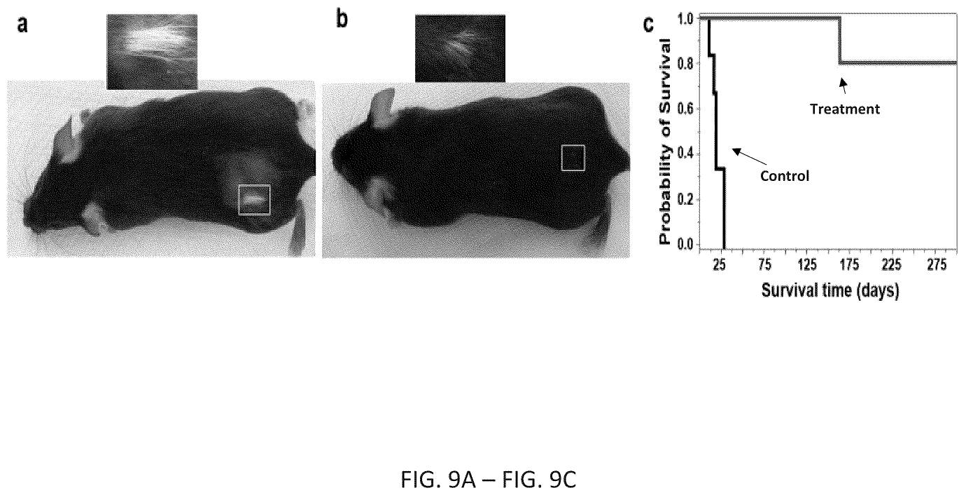

Some mice developed depigmented hairs at melanoma treatment sites indicating localized immunotargeting of the tyrosinase+ melanocytes in these regions (FIG. 9A). Within 100 days from the beginning of the experiment, treated mice did not develop secondary lesions. Mock-treated animals perished within 30 days (FIG. 9C). At day 100, all experimental animals received a challenging inoculation of the B16/A2 tumors. These secondary lesions were rejected. Similar to the initial treatment, depigmented hairs were detected at sites of challenging inoculation. All treated mice receive another challenging inoculation at day 200, which was also rejected. All experimental animals lived until day 300. Some of them died at this time because of advanced age. Others were euthanized for collection of splenocytes. Collectively, these in vivo studies demonstrated that intratumoral CCL21 priming combined with in vivo electroporation of the Tyr-TCR and ΦC31-encoding plasmids resulted in activation of the melanoma-specific CTLs capable of killing antigen-positive tumors, localized autoimmunity, and generation of melanoma-specific immunologic memory allowing rejection of secondary and tertiary lesions.

### Example 2: Targeting of Tumor Lesions Via Non-Viral In Vivo Genetic Engineering of the Tumor-Reactive T Cells

The technology described herein represents a transformational, high-impact initiative at genetic engineering of the tumor-reactive T cells as a new type of cancer vaccine where patients are directly treated via in vivo plasmid DNA transfer to generate recombinant tumor-reactive T cells capable of targeting and eliminating malignant lesions. It represents a conceptually novel and versatile platform for TCR and CAR-based T cell therapies. The experiments described herein are developed to utilize innovative concepts of the proposed strategy: (i) Chemokine-mediated recruitment of specific T cell populations to the gene transfer sites via engagement of the specific chemokine receptors allows maximizing tumoricidal activity and generation of immunologic memory; (ii) development of approaches to enhance T cell egress from the gene transfer sites ensures T cell activity migration to systemic compartment and distal tumor lesion; (iii) structural modifications to the recombinant TCR are aimed at improvement of the tumoricidal capacity, proliferation, viability and persistence of tumor-reactive TCR-T cells allowing achieving desirable efficacy with smaller number of tumor-reactive T cells; (iv) ΦC31-integrase-mediated genetic recombination permits gene transfer into quiescent naïve and central memory T cells and reduces risk of insertional mutagenesis by mediating integration of TCR and CAR expression cassettes into specific genomic sites known as pseudo-attP sites; (v) Plasmid-based mammalian expression system simplifies plasmid construction and allows cell type specific promoters to restrict transgene expression; (vi) in vivo DNA transfer via electroporation permits efficacious gene transfer in animals and human patients (Trimble et al., Lancet 2015, 386:2078-2088); (vii) Tumor reactive T cells could be genetic engineering to express different targeting molecules (TCRs and CARs) in a single procedure; (viii) non-viral in vivo gene transfer utilizes low-cost (compared to virus) cGMP grade plasmid DNA and eliminates ex vivo manipulations with patient-derived T cells making this approach cost-effective and adaptable for widespread utility.

### T Cell Recruitment to the Gene Transfer Sites.

T cell genetic engineering in vivo requires availability of the T cells at gene transfer sites. As demonstrated by previous studies, it could be achieved by forced expression of the chemokines in normal skin and malignant lesions (Igoucheva et al., Gene therapy 2013, 20:939-948; Igoucheva et al., Oncoimmunology 2013, 2: e26092; Kemp et al., Oncotarget 2017, 8:14428-14442; Novak et al., Molecular cancer therapeutics 2007, 6:1755-1764). Chemokines not only enhance T cell trafficking but also can selectively recruit specific T cells populations by engaging specific chemokine receptors. For example, CCL17 and CCL22 preferentially mediate extravasation of the peripheral memory and effector CCR4+ T cells, CCL27 mediates migration of the CCR10+ T helper (Th) cells within the skin, and CCL21 enhances extravasation and migration of CCR7+naïve and central memory T cells (TCM), whereas CCL5 mediates recruitment of CCR4 and CCR5 CD4+Th1 and CD8+ cytotoxic lymphocytes (CTL). Although it was shown that CD4+ and CD8+ T cells are required for effective recombinant TCR-T cell based therapies (Burns et al., Cancer research 2010, 70:3027-3033) and that T_{CM} could be advantageous for tumor immuno-targeting (Kueberuwa et al., Journal for immunotherapy of cancer 2017, 5:14), selection of specific T cell populations for gene transfer remains underdeveloped.

The experiments provided herein further explore chemotactic T cells recruitment, characterize responding T cell populations and select approaches maximizing in vivo gene transfer, and investigate T cell egress from gene transfer sites and tumoricidal capacity of the engineered T cells.

To improve T cell recruitment to the vaccine administration site, additional mammalian expression vectors encoding inflammatory and constitutive chemokines including CCL2, CCL5, CCL20, CCL21, CCL22, CCL27, and CXCL12 could be added to the pre-conditioning protocol. The current (FIG. 5) and prior (Igoucheva et al., Gene therapy 2013, 20:939-948; Igoucheva et al., Oncoimmunology 2013, 2: c26092; Kemp et al., Oncotarget 2017, 8:14428-14442; Novak et al., Molecular cancer therapeutics 2007, 6:1755-1764) data demonstrate that transient CCL21 expression in the skin and tumor lesions enhances infiltration of these tissues with T cells and suggest that combination of CCL21 with other chemokines could further improve T cell recruitment to gene transfer sites. To test this, experiments are conducted with a CD4-GFP transgenic mouse model (JAXmice, stock #008126) in which more than 80% of naive or resting CD4+ and CD8+ T cells uniformly express GFP (Manjunath et al., Proceedings of the National Academy of Sciences of the United States of America 1999, 96:13932-13937). The experimental cohort of mice is treated with CCL21 alone or in combination with CCL17, CCL5 and CCL22 via electroporation of the chemokine-encoding plasmids under previously optimized conditions (Igoucheva et al., Gene therapy 2013, 20:939-948). Mice are treated with chemokines at 4 sites per animal. To ensure chemotactic T cell recruitment, an additional cohort of mice is pretreated for 2 days prior to electroporation with pertussis toxin (PTX) to inhibit T cell chemotaxis (Chen et al., European journal of immunology 2006, 36:671-680. 3153960). Semi-quantitative comparison of GFP+ T cell recruitment to the chemokine-primed skin is done using IVIS imaging system for 8 days with 2-day intervals. At each time point, tissue samples are collected from sentinel mice for immunofluorescent and FACS analyses to quantify skin/tumor-infiltrating GFP+ T cells. Populations of the recruited T cells, recovered from the tissues are also analyzed by FACS using T cell subset specific markers (Farber et al., Nature reviews Immunology 2014, 14:24-35. 4032067). Similar treatments are done in pre-established intradermal melanoma and lymphoma lesions (2 lesions per mouse). Because these tumor models are established in syngeneic C57BL6/HLA-A2 mice (further referred to as HLA-A2 mice) and in wild-type C57BL6, analysis of tumor-infiltrating cells is done by indirect immunofluorescent on cryosections and by FACS using T cell and subset-specific antibody.

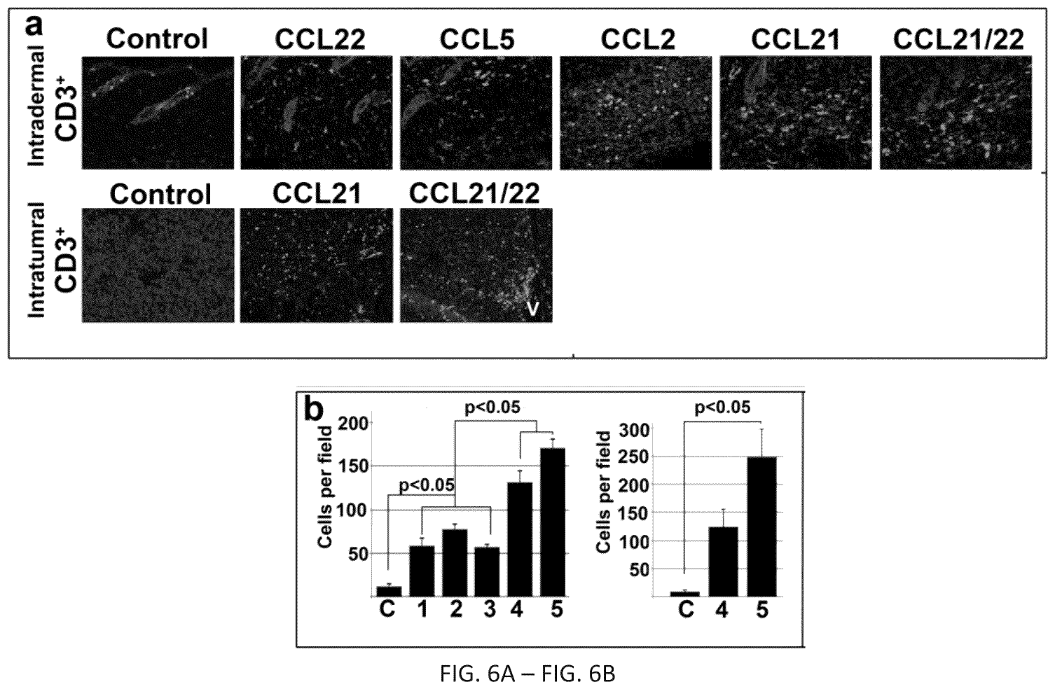

To better define T cell response to specific chemotactic signals, mammalian expression vectors encoding inflammatory and constitutive chemokines including CCL2, CCL5, CCL20, CCL21, CCL22, CCL27, and CXCL12 were generated. T cell recruitment to the skin and experimental intradermal melanoma lesions were assessed 72 h after electroporation of these plasmids. CXCL12, CCL20, CCL27 alone failed to appreciably alter T cell recruitment to the skin and tumor lesions (data not shown). Expression of CCL5 and CCL22 increased T cell infiltration of the skin 5-7-fold as compared to control, whereas CCL2 substantially increased infiltration of the skin with myeloid cells. Treatment of the skin with CCL21 alone or in combination with CCL22 increased recruitment of the T cells about 11-13-fold. The latter treatment was particularly effective in the intradermal melanoma, improving infiltration of the lesions with T cell up to 30 times likely due to the presence of the well-established intratumoral blood vessels (FIG. 6A and FIG. 6B).

T Cell Egress from the Gene Transfer Sites.

Efficient T cell exit from the gene transfer sites via afferent lymphatic and draining lymph nodes (LN) into blood circulation is important for a wide distribution of the engineered recombinant T cells, generation of immunologic memory and immunotargeting of distal tumor lesions. Previous studies have demonstrated that T cell egress from extralymphoid tissues, particularly from the skin, is tightly regulated by the chemokine receptor CCR7 (Jennrich et al., Journal of virology 2012, 86:3436-3445. 3302526; Debes et al., Nature immunology 2005, 6:889-894), which drives T cells to afferent lymphatic, known to constitutively secrete the CCR7 ligand-CCL21 (Russo et al., Cell reports 2016, 14:1723-1734; Hunter et al., Frontiers in immunology 2016, 7:613). Since it is expected that CCL21-mediated recruitment of the T cells to the gene transfer sites will selectively recruit CCR7+ CD62L+ T cells, it is anticipated that these cells retain the ability to egress to lymphatic system and to blood circulation after gene transfer and proteolysis of transiently expressed CCL21. This notion is indirectly supported by current data (FIG. 9) showing acquisition of immunologic memory by tumor-rejecting mice. To better define T cell egress from the cutaneous and tumor tissues after in vivo gene transfer, draining LN, spleen and blood of sentinel mice treated with Tyr-TCR-DsRed and CD19-CAR-EGFP is collected. Number and phenotype of DsRed+ and EGFP+ T cells migrated to the secondary lymphoid organs and the blood is assessed by FACS-based immuno-phenotyping using subset-specific surface markers (Farber et al., Nature reviews Immunology 2014, 14:24-35. 4032067). Repertoire of the chemokine receptors (Alexeev et al., Stem cell research & therapy 2016, 7:124) and selectins expressed on recovered T cells is also analyzed.

### Improvement of T Cell Functional Activity Via Structural Modification of the Recombinant TCR.

To date, several melanoma-specific TCR-T cells were shown to eradicate bulky malignant lesions in stage IV melanoma patients after ACT (Park et al, Trends in biotechnology 2011, 29:550-557; Phan et al., Cancer control: Journal of the Moffitt Cancer Center 2013, 20:289-297; Ruella et al., Current hematologic malignancy reports 2016, 11:368-384; Frankel et al., J Immunol 2010, 184:5988-5998; Cohen et al., Cancer research 2007, 67:3898-3903; Perro et al., Gene therapy 2010, 17:721-732). However, it was established that recombinant α/β TCRs, although selected for high affinity binding, do not transmit proper intracellular signals when ligated to Ag resulting in reduced viability, proliferation and tumoricidal activity. To enhance T cell functional activity, addition of CD35, CD137 (4-1BB) and CD28 signaling domains were employed in CAR design. These signaling elements allowed T cells to operate independently of MHC engagement and augmented cytokine production, antitumor activity and viability of the CAR-T cells (Finney et al., J Immunol 2004, 172:104-113; Milone et al., Molecular therapy: The Journal of the American Society of Gene Therapy 2009, 17:1453-1464; Ramos et al., Expert opinion on biological therapy 2011, 11:855-873. 3107373). Addition of these domains to the recombinant TCR was not tested, however, such modifications could be advantageous for the in vivo T cell genetic engineering where a smaller number of recombinant T cells could support effective tumor immunotargeting.

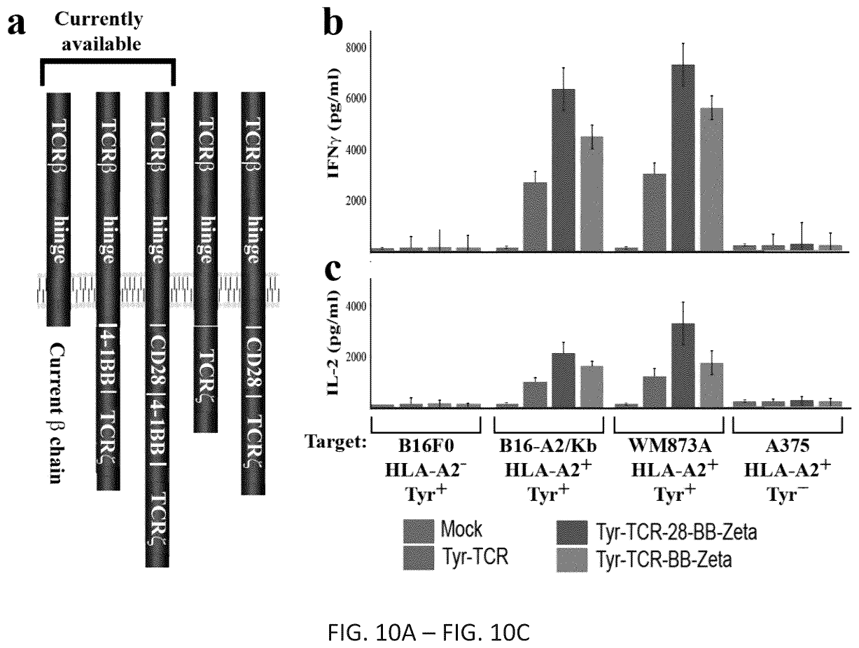

cDNA encoding CD35, CD137 (4-1BB) and CD28 signaling domains were obtained and were ligated in different combinations in frame with cDNA encoding B chain of the a/β Tyr-TCR yielding in structurally different constructs (FIG. 10A). Freshly isolated pan T cells (mouse and human) were transduced with generated constructs via nucleofection (Lonza) in vitro and cultured for 24 h. Then, T cells were exposed to irradiated mouse B16F0 (HLA-A2−), B16/A2 (HLA-A2+Tyr+), or human WM983 (HLA-A2+Tyr+) and A375 (HLA-A2+Tyr−) melanoma cells for 48 h. IFNγ and IL-2 production was measured by ELISA. As compared to the original Tyr-TCR, T cells expressing CD28-CD137-CD3ζ (28BBZ)- and CD137-CD3ζ (BBZ)-modified TCRs produced greater quantities of both cytokines when exposed to WM983 (human) or B16/A2 (mouse) cells (FIG. 10B). Augmented secretion of type 1 cytokines suggested that these structural modifications could improve tumor-targeting capacity of the recombinant TCR-T cells and provide better tumor targeting with smaller number of the recombinant T cells.

To further evaluate the contribution of the proposed structural modifications, primary human T cells isolated from PBMC are transduced with different Tyr-TCR constructs via ΦC31-integrase mediated gene transfer under established conditions. T cells are stimulated with Tyr368 376 peptide and exposed to HLA-A2+, Tyr+ and HLA-A2+, Tyr-melanoma cells. Population doublings are assessed by standard CFSE dilution assay, every day for 6 days by FACS. Production of type 1 (IL-2, IFNγ, IL12) and type 2 (IL-4, IL-10, IL-6) cytokines are examined by LegendPlex Multianalyte Flow ELISA (Biolegend). Survival of T cells transduced with different constructs are assessed by culturing T cells in the absence or presence of Tyr+HLA-A2+ targets without IL-2 for 3 weeks. Every 4 days, T cell viability are examined by FACS-based assay (Millipore). Lytic activity of the T cells expressing different TCR constructs are assessed by Granzyme B activity assay and fluorescence-based CTL assay as described previously (Igoucheva et al., Gene therapy 2013, 20:939-948; Novak et al., Molecular cancer therapeutics 2007, 6:1755-1764). Comparative analysis identifies most potent TCR structure that could support durable antigen-specific CTL response in vivo.

### Analysis of Intradermal and Intratumoral Gene Transfer Efficacy.

After selecting T cell recruitment protocol, 3 cohorts of HLA-A2 animals are primed with chemokines intradermally. Experimental and control cohorts receive chemokine(s) in 4 sites and additional experimental cohort are primed with chemokine(s) at 1 site. After 48 h, primed sites are electroporated with Tyr-TCR-DsRed construct (described above) and gene transfer efficacy is evaluated by reporter (Ds-Red) expression using IVIS live animal imaging. At three day intervals over a 12 day period, sentinel mice from each cohort are euthanized and DsRed expression in T cells are assessed on cryosections by indirect immunofluorescence and by FACS on skin-recovered T cells. Expression of general and subset-specific T cell markers including CCR7 are analyzed. Tyr-TCR expression in DsRed+ cells are also examined using Tyr368-376-specific tetramers.

To validate gene transfer efficacy in settings of established B16/A2 and 38c13 lesions, similar gene transfer experiments are set up. After priming with chemokine(s), B16/A2 lesions are electroporated with the Tyr-TCR-DsRed construct, whereas 38c 13 lesions are transduced with the CD19-CAR-EGFP construct (described elsewhere herein). In all gene transfer studies, a plasmid encoding ΦC31 integrase are co-electroporated in established transgene: integrase ratio to provide efficient genomic integration. Further analysis of the gene transfer is conducted as described for the intradermal sites.

### Analysis of T Cell Populations Suitable for Genetic Engineering/Re-Programming In Vivo.

Prior data demonstrated that CCL21 preferentially recruits CCR7+ naïve T cells and T_{CM} (Igoucheva et al., Gene therapy 2013, 20:939-948; Novak et al., Molecular cancer therapeutics 2007, 6:1755-1764). These two populations could be most advantageous for the in vivo gene transfer: the former could produce a large number of effector T cells, whereas the latter could rapidly proliferate and differentiate into effector and memory T cells. It is expected that these two populations will be present at the gene transfer sites. However, it is well established that the majority of the tissue-residing and circulating T cells are antigen-experienced lymphocytes that take part in immune surveillance. It is likely that these cells will be the primary responders to the altered chemotactic gradients within the skin and malignant lesions. Although redirection of the T cells by TCR and CAR was shown in ACT studies, it is still not well-defined whether previously primed T cells could be effectively redirected to new antigen (Ag) by forced expression of the recombinant TCR or CAR. To investigate this, CD8-restricted OT-I T cells expressing ovalbumin-specific TCR recognizing the SIINFEKL peptide are employed. In the in vitro studies, OT-I cells are transduced with Tyr-TCR, cells are re-activated with Ova or Tyr peptides, and T cell activity against B16-Ova and B16/A2 cells is assessed. IFNγ-ELISpot and CTL assays allow for determining whether OT-I cells could be effectively redirected by Tyr-TCR to tyrosinase+ HLA-A2+ B16/A2 cells.

### Analysis of ΦC31-Integrase Mediated Genomic Integration.

Currently, retrovirus-mediated gene transfer is common for recombinant T cells production ex vivo (Park et al, Trends in biotechnology 2011, 29:550-557). Lentiviral and Sleeping Beauty transposon systems were also tested for T cell engineering (Frecha et al., Molecular therapy: The Journal of the American Society of Gene Therapy 2010, 18:1748-1757. 2951569; Peng et al., Gene therapy 2009, 16:1042-1049). It is examined herein whether the ΦC31-integrase-mediated gene transfer is more advantageous for in vivo applications. ΦC31-integrase-mediated gene transfer allows: (i) genetic manipulations with quiescent T cells; (ii) minor alteration of the expression vectors encoding TCR or CAR (ligation of an attB sequence) and (iii) predefined and preferential genomic integration of expression cassettes with transgenes into 3 specific sites known as pseudo attP sites in the human genome located in Xq22.1, 8p22, 19q13.31 loci (Groth et al., Proceedings of the National Academy of Sciences of the United States of America 2000, 97:5995-6000).

To assess the capacity of the non-viral ΦC31 integrase-mediated gene transfer, freshly isolated human T cells were co-transduced with EGFP-attB and ΦC31-integrase plasmids. Nucleofection of the T cells resulted in the expression of the transgene (EGFP) in 23% and 38% of CD4+ and CD8+ T cells, respectively (FIG. 4A and FIG. 4B). Stimulation of T cells for 2 weeks led to a 40-fold expansion of culture and durable EGFP expression in 70% of the T cells (FIG. 4C and FIG. 4D). Human T cells transduced with Tyr-TCR and ΦC31-integrase under these conditions showed high CTL activity against Tyr+ melanoma cells in vitro (FIG. 4E) with ˜35% of CD8+ T cells showing binding to Tyr368-376 specific tetramers (iTAg-MHC tetramer, MBL) (FIG. 4F). When compared to γ-rertoviral gene transfer (Frankel et al., J Immunol 2010, 184:5988-5998), ΦC31-mediated integration produced 2 times more recombinant T cells. These data confirmed that ΦC31-mediated gene transfer provides durable Tyr-TCR expression and production of functional CTL.

To better characterize ΦC31-mediated recombination, human T cells are transduced with Tyr-TCR and ΦC31 integrase at different ratios (5:1; 10:1; 20:1). Cells are propagated up to 6 weeks and T cells are examined for the genomic integration into psudo-attP sites by genomic DNA specific PCR as described previously (Groth et al., Proceedings of the National Academy of Sciences of the United States of America 2000, 97:5995-6000) and for the cell-surface expression of TCR by Tyr368-376 tetramers as described (Frankel et al., J Immunol 2010, 184:5988-5998). In similar settings integration of melanoma-specific CSPG4-CAR and lymphoma-specific CD19-CAR is assesed.

To validate durability of ΦC31 integrase-mediated genetic engineering after in vivo gene transfer, T cells recovered from the tissues are cultured in vitro with CD3/CD28 and IL-2 stimulation and analyzed by FACS for the expression of the transgenes (TCR and CAR) and reporters (DsRed and EGFP) for 60 days at 10 day intervals.

The experiments presented herein allow for the identification of optimal combination of chemokines for the efficient intralesional and intradermal recruitment of the T cells, determination of the T cell populations responding to these chemokines, evaluate gene transfer efficacy and durability of the in vivo engineered T cells and, possibly, the improvement of recombinant TCR-T cell activity via structural modifications to achieve more effective tumoricidal capacity with a smaller number of cells. The experiments also investigate whether intradermal treatment is more efficient in generating recombinant, tumor-targeting T cells than the intratumoral treatment. While not wishing to be bound by any particular theory, intradermal treatment may be more efficient because the current data shows that T cells represent the majority of intradermal cells at chemokine-primed sites and that high localized density of these cells creates favorable conditions for the efficacious gene transfer.

To optimize in vivo gene transfer, an expression vector in which expression of a transgene is controlled by T cell-specific CD38 promoter and regulatory elements is established. Constructs encoding Tyr-TCR, CD19-CAR and CSPG4-CAR are created using this vector.

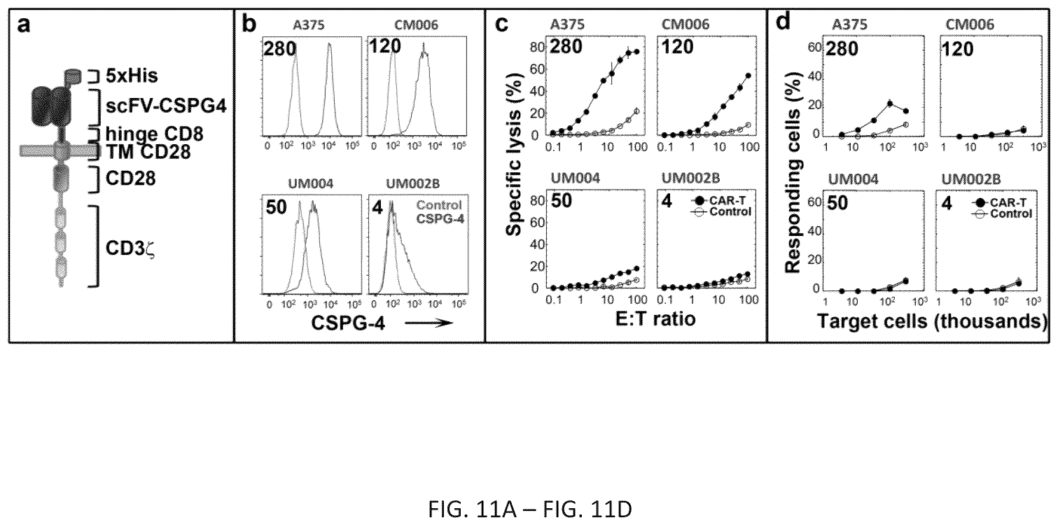

Previous studies indicate that CD4+ T cells egress is on average 3 to 12 fold more efficiently than CD8+ T cells and the data may indicate a necessity to assess longer time points, additional cohorts of animals are used. Previous studies also demonstrated that Ag-experienced T cells with high antigenic load down-modulate CCR7 as relevant to viral infection (Jennrich et al., Journal of virology 2012, 86:3436-3445. 3302526). This phenomenon could be advantageous for the intratumoral treatment, as observed in the data presented herein (FIG. 11). In certain instances, if or when downmodulation of the CCR7 is detected after intracutaneous gene transfer, CD38-CCR7 plasmid (without attB site) may be added to the treatment to provide transient expression of this chemokine receptor in genetically engineered T cells to maximize egress to afferent lymphatics and circulation.

To develop a tracing system that permits detection of the recombinant T cells at metastatic sites in vivo, mutant HSV1-sr39tk cDNA is linked with Tyr-TCR and CD19-CAR constructs via P2A element. Translation of receptors and mtHSV1-sr39tk from a single open reading frame permits non-invasive monitoring of T cells in metastatic lesions by PET imaging with nucleoside-based probes (Gambhir et al., Proceedings of the National Academy of Sciences of the United States of America 2000, 97:2785-2790. 16007. Munoz-Alvarez et al., Molecular therapy: The Journal of the American Society of Gene Therapy 2015, 23:728-736).

### Example 3: Evaluation of the Tumoricidal Capacities of the In Vivo Engineered TCR- and CAR-T Cell

To date, recombinant TCR and CAR-modified T cells showed remarkable success in clinical settings (Park et al, Trends in biotechnology 2011, 29:550-557). Recently, CD19-CAR-T cells were approved by the FDA for the ACT treatment of B-cell acute lymphoblastic leukemia (First-Ever CAR T-cell Therapy Approved in U.S, Cancer discovery 2017, 7:OF1). However, several drawbacks restrict broad application of both strategies. For example, CAR-T cells require tumor-associated Ag expression on the surface of malignant cells and, at large, were effective only against liquid tumors. TCR-T cells, while recognizing peptides derived from virtually all cell-expressed proteins and eliminating solid tumors, are restricted by MHC presentation and convey considerable on-target and off-target toxicities. In vivo genetic engineering offers a unique opportunity to conduct side-by-side comparative studies to better define pros and cons of both approaches, evaluate tumoricidal capacity of TCR and CAR in nearly identical experimental conditions in settings of established tumor lesions

To test the capacity of the in vivo T cell genetic engineering in a tumor-bearing host, ˜100 mm³ B16/A2 intradermal melanomas were established in HLA-A2 transgenic mice. Lesions were primed with CCL21 to recruit T cells following in vivo electroporation of the Tyr-TCR- and ΦC31-encoding plasmids. After gene transfer procedure, about 50% of the T cells recovered from treated lesions showed Tyr-TCR expression on cell surface, as defined by Tyr-TCR-specific tetramer staining (FIG. 7B). Analysis of the intratumoral CD4+ and CD8+ T cells showed that about 50% of T cell types expressed Tyr-TCR (FIG. 7B). Recovered cells present B16/A2-specific cytotoxic activity as measured by the in vitro CTL assay (FIG. 7C) Four consecutive treatments led to a complete remission of the intradermal lesions in 70% of experimental animals (3 independent experiments, 6 animals per experiment) and substantial reduction of tumor burden in other animals (FIG. 8A-FIG. 8C). All treated mice developed local depigmentation at treatment sties suggesting immunotargeting of normal melanocytes (FIG. 9A) albeit at a substantially lower extent then in mice and melanoma patients treated via ACT of the Tyr-TCR-T cells (Phan et al., Cancer control: journal of the Moffitt Cancer Center 2013, 20:289-297; Frankel et al., J Immunol 2010, 184:5988-5998). As no secondary lesions were detected in tumor-rejecting animals within 100 days, these mice received challenging inoculation of the B16/A2 cells into opposing flanks, which were completely rejected. Local depigmentation at sites of challenging inoculation was also observed. (FIG. 9B) All tumor-rejecting animals lived until day 300 with no signs of tumor development (FIG. 9C). Tumor lesions were not detected during pathological evaluation on sacrifice. Collectively, these studies demonstrated the ability of the in vivo engineered T cells to eliminate established tumor lesions and generate long lasting protective immunologic memory, thus confirming the feasibility of this novel technology.

### Utility of the In Vivo T Cells Genetic Engineering for the Analysis of Tumor-Targeting Constructs.

To evaluate whether the present technology could be utilized for rapid evaluation of the tumor-targeting molecules directly in tumor bearing host, the tumoricidal activity of the T cells engineered in vivo to express the original and the modified Tyr-TCR constructs (FIG. 10A) are compared. B16/A2 cells are metabolically labeled with DiO green fluorescent tracer (Invitrogen) and inoculated into both flanks of syngeneic mice (experimental and control cohorts). Lesions are primed with chemokines under optimized conditions and experimental cohorts are treated with Tyr-TCR constructs and ΦC31 integrase under optimized conditions. Activity of different constructs are compared by assessing tumor growth inhibition in a 4-week period (endpoint). After each week, sentinel mice are euthanized and lesions are evaluated for the presence of proliferating and apoptotic malignant cells by indirect immunofluorescence as previously described (Kemp et al., Oncotarget 2017, 8:14428-14442). Metabolic labeling of the B16/A2 cells allows for the outline of malignant cells and estimation of their killing by FACS. Intratumoral granzyme B activity, as a “reporter” of the cytolytic T cell activity is measured on tumor lysates using fluorescence-based granzyme B activity kit (Sigma-Aldrich). Cytolytic activity is also estimated by the analysis of the T cell degranulation in situ by the indirect immunofluorescent detection of the CD3/CD107a on cell surface using confocal microscopy. Intratumoral T cells are recovered from single cell suspensions of tumors using positive selection and used for quantitation of Tyr-TCR+ cells by FACS-based tetramer staining, in vitro CTL activity against B16/A2 and B16F10 cells and proliferative capacity. Similar assessments are conducted at the endpoint. Combined data allows for the determination of whether addition of signaling domains augments recombinant T cells activity in vivo and define conditions for rapid evaluation of the tumor-targeting receptors in settings of established tumors in vivo.

### Melanoma-Targeting Capacity of CAR-Modified T Cells.

To date, mechanistic factors that limit sensitivity and magnitude of the CAR-T cell response to solid tumors remain incompletely understood. In vitro studies suggested that the imbalanced CAR:antigen ratio required to satisfy the frequency threshold of receptor-ligand interactions to achieve effector cell activation could be one of the key limiting factors (Oren et al., J Immunol 2014, 193:5733-5743). However, in vitro studies cannot recapitulate the complexity of the tumor microenvironment, which may positively or negatively affect T cells, whereas in vivo studies aimed at sequestration of limiting factors using ACT are not feasible as they could be time-consuming and labor retaining. T cell genetic engineering in vivo offers a unique opportunity to address some of these important mechanistic questions directly in settings of established tumor lesions.

To define tumoricidal capacity of the CAR-T cells in melanoma settings, a CAR construct that recognizes chondroitin sulfate proteoglycan 4 (CSPG4) (high molecular weight melanoma-associated antigen (HMW-MAA) could be used. CSPG4 is expressed on greater than 90% of the human melanomas (Beard et al., Journal for immunotherapy of cancer 2014, 2:25. 4155770) and could be targeted by CAR-T cells in vitro and in vivo (Burns et al., Cancer research 2010, 70:3027-3033). The established CSPG4-CAR construct could be used in in parallel with experiments using Tyr-TCR and both approaches could be compared side by side.

The established CSPG4-CAR construct contains an extracellular single chain Fv antibody specific to CSPG4 with His tag and intracellular CD28 and CD38 signaling domains (FIG. 11A). Human T cells, stably expressing this CSPG4-CAR demonstrated differential cytolytic activity to human melanoma cell lines in vitro. The extent of specific lysis by the CAR-expressing effector cells was greatly influenced by the expression level of CSPG4 on the tumor cell surface (FIG. 11B and FIG. 11C). This trend was confirmed by the assessment of T cell degranulation, as detected by the cell surface exposure of the CD107a (FIG. 11D). Only A375 cells with the highest number of cell-surface antigens could induce effective granule release by CAR T cells. No other examined tumor cells induced substantial degranulation of the CAR-T cells suggesting that granule release is heavily influenced by ligand concentration.

To validate these findings in settings of the established melanoma lesions, B16/A2 and B16F10 cells are transduced with CSPG4 as described in prior studies (Maciag et al., Cancer research 2008, 68:8066-8075) and high and low CSPG4-expressing clones are selected. Cell-surface CSPG4 is estimated by FACS using fluorescently-labeled beads as advised by the QuickCal protocol (Bangs Labs Quantum MESF kits). Full length attB sequence for the ΦC31 integration is ligated into the expression vector coding for the CSPG4-CAR. Further, B16/A2-CSPG4 high and low expressing cells are inoculated into left and right flanks of the HLA-A2 mice. Established lesions are primed with chemokine(s) and treated with CSPG4-CAR and ΦC31 encoding plasmids under optimized conditions. One cohort of animals is euthanized 48 h after treatment to isolate intratumoral T cells and semi-quantify CSPG4-CAR expression on T cells using QuickCal protocol (Bangs Labs). Remaining cohorts are treated and analyzed as described above.

### Comparative Analysis of Tumoricidal Capacity of the TCR- and CAR-Modified T Cells.

The majority of the current studies aimed at comparing CAR and TCR cytolytic capacities against solid tumor-derived malignant cells have demonstrated that TCR-T cells have higher cytolytic activity including studies where CAR and TCR constructs were selected to recognize same antigenic complex (Oren et al., J Immunol 2014, 193:5733-5743). Although being instrumental in defining limiting factors, these studies did not account for the complexity of the tumor environment. Intratumoral T cell genetic engineering allows us to evaluate tumoricidal capacity of the TCR- and CAR-modified T cells directly in tumor-bearing hosts in virtually identical experimental settings in vivo. To conduct this comparison, CAR-T cell sensitive B16/A2-CSPG4 tumor lesions are established in flanks of HLA-A2 transgenic mice. Right and left lesions are treated with Tyr-TCR and CSPG4-CAR, respectively, under established conditions. Tumor growth monitoring and analysis of the tumoricidal capacities of the Tyr-TCR and CSPG4-CAR are carried out as described above.

### Intradermal Genetic Engineering of the Melanoma-Targeting Recombinant T Cells.

As demonstrated by the data presented herein, intratumoral genetic engineering could serve as a tool to evaluate T cell-targeting constructs such as TCR or CAR in settings of established tumor lesions and could be clinically applicable for the treatment of the unresectable lesions during surgery, treatment of several intracutaneous malignancies or, with the development of the ultrasound-guided gene delivery tools, for the direct treatment of metastatic lesions. However, development of a more broadly applicable approach is desirable. As demonstrated by the data presented herein, intradermal T cell recruitment and gene transfer could be utilized for this purpose (FIG. 9). Yet, it is not known, whether: (i) sufficient number of the recombinant T cells could be generated and whether (ii) T cell egress from the skin to the systemic compartment and distant lesions will permit effective tumor targeting. To further elucidate the ability of the intradermally engineered T cells to target distal metastases, a B16/A2 pulmonary metastasis model is employed. B16/A2 cells are injected intravenously as previously described (Igoucheva et al., Gene therapy 2013, 20:939-948) into 2 cohorts of HLA-A2 mice. After 1 week, intradermal sites of these animals are primed with chemokines and experimental cohort are treated with Tyr-TCR and ΦC31 constructs under optimized conditions. Treatments are repeated 4 times with one week interval. Each week, up to the endpoint (5 weeks from the first treatment), sentinel mice from experimental and control cohorts are euthanized and pulmonary metastases are examined and enumerated. T cells isolated from draining lymph nodes, blood and a single cell suspension of the excised cumulative metastatic lesions are analyzed for the presence of the Tyr-TCR+ T cells by FACS using Tyr-TCR-specific tetramers (iTAg-MHC tetramer, MBL). Splenocytes are also analyzed for the presence of the Tyr-TCR+ T cells by FACS and by IFNγ ELISpot assay against B16/A2 and HLA-A2.1-negative parental B16F10 targets.

### Targeting of B Cell Lymphoma by the In Vivo Engineered CD19-CAR-T.

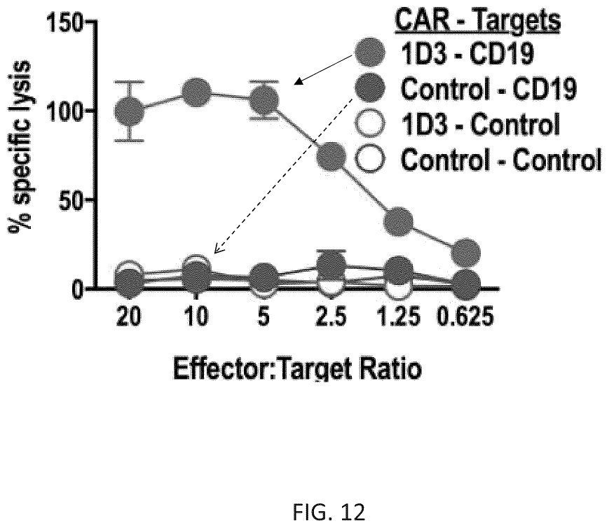

ACT with CAR-T cells specific to CD19 were shown to effectively target B cell malignancies and were recently approved by the FDA for the treatment of B-cell precursor acute lymphoblastic leukemia (First-Ever CAR T-cell Therapy Approved in U.S, Cancer discovery 2017, 7: OF1). It is suggested that the success of the CD19-CAR T cells in targeting B cell malignancies is associated with the abundance of the CD19 expression on the surface of the B cells and the relative accessibility of the malignant cells to the CAR-T cells. Studies on animal models showed that i.v. injection of the transformed B cells into wild type mice leads to the development of the pancytopenia and death from bone marrow (BM) failure and that injection of the CD19-CAR T cells (1×10⁷) improves survival of mice. This study also approximated that a ratio of 1 CD19-CAR T cell to 12 malignant B cells is needed for persistent CAR-T cell function (Davila et al., PloS one 2013, 8:e61338). To initiate studies aimed at targeting CD19, 3rd generation CD19-CAR constructs using the 1D3 antibody sequence with the 5×His tag, CD35, CD28 and 4-1BB domains, and inactivated 1st and 3rd ITAMS of CD35 and modified CD28 (LL-GG) is used. T cells transduced with this construct showed high cytolytic activity against CD19-positive targets (FIG. 12). An additional construct containing CD19-CAR with IRES-linked GFP cassette for fluorescent detection of CD19-CAR-T cell is also used.

To test whether intradermal in vivo gene transfer produces functional CD19-CAR T cells, which can target B cell lymphoma in systemic compartment, 2 cohorts of the wild type C57BL6 mice receive an i.v. injection of the B cell lymphoma cells (38C13 cells). After 5 days, experimental mice receive 4 consecutive treatments with CD19-CAR (without GFP cassette), once a week for 4 weeks. Each week, blood samples from all mock-treated and experimental mice is collected and white cell count, hemoglobin, and platelets are measured and statistically analyzed. Presence of the B cell lymphoma cells and CD19-CAR-T cells in the blood is evaluated. CD19-CAR-T cells is assessed by FACS using 5×His tag, whereas B cell lymphoma cells are discriminated from the normal B cells by its aberrant phenotype (cell surface expression of the k light chain and CD19 but not B220) as described previously (Kochenderfer et al., Blood 2010, 116:3875-3886). Each week sentinel mice are euthanized and blood, bone marrow and spleen are harvested for anatomical and cellular analyses, assessment of pancytopenia, and presence of leukemic B cells and CD19-CAR T cells in BM. All data obtained from experimental animals are compared to mock-treated control.

The experiments presented herein allow for the evaluation of the utility of the proposed technology for a comparative analysis of the T cell-targeting molecules, such as TCR and CAR, to test whether addition of the signaling domains to the recombinant TCR structure enhances tumoricidal capacity of the recombinant T cells and, if so, define which domains are essential for these improvements. The experiments also define the utility of melanoma-specific CAR-T cells in targeting solid tumors. Moreover, the experiments evaluate the capacity of the intradermally engineered T cell and the ability of the CD19-CAR T cell to target B cell lymphoma. Experiments may also be conducted to use multiple skin sites for in vivo gene transfer and to evaluate the efficacy of intradermally delivered CSPG4-CAR-encoding constructs. Further, experiments may be conducted using 3rd generation CSPG4-CAR construct (similar to current CD19-CAR) by re-ligating all 3 signaling domains and investigating whether addition of CD137 (4-1BB) improves melanoma-targeting capacity of this CAR. For comparison of different TCR constructs, Nur77-GFPCre BAC transgenic mice (JAXmice, stock #018974) can be used, in which the level of GFP expression reflects the strength of TCR stimulation. These mice may be bred to HLA-A2 transgenic animals to provide an alternative readout assay to compare the activity of the recombinant T cells expressing human HLA-A2-restricted TCR (such as Tyr-TCR) in vivo after gene transfer and follow activated T cells based on GFP expression.

### Example 4: Investigation of the Therapeutic Utility of the In Vivo Engineered TCR- and CAR-Modified T Cells in Immunotargeting of Established Solid and Liquid Tumors

In vivo T cell genetic engineering offers several advantages over ACT including the ability of multiple treatments, rapid change of treatment regimen, concurrent or consecutive engineering of the recombinant T cells expressing different tumor-specific molecules (e.g. Tyr-TCR and CSPG4-CAR), or co-targeting of specific malignant cell populations with different CAR or TCR-modified T cells (Schmidt et al., Proceedings of the National Academy of Sciences of the United States of America 2011, 108:2474-2479). The experiments presented herein evaluate its therapeutic capacity of in vivo engineered TCR- and CAR-modified T cells to target solid (melanoma) and liquid (B cell lymphoma) tumors in clinically relevant tumor settings.

Several types of more aggressive skin malignancies such as cutaneous diffuse large B-cell lymphoma and superficial spreading melanoma with multiple nodules present a substantial challenge: these lesions often cannot be treated locally with radiation or surgery and systemic treatment, although applicable (Kochenderfer et al., Journal of clinical oncology: official journal of the American Society of Clinical Oncology 2015, 33:540-549), may present considerable toxicity which could outweigh the benefits of the treatment. Such malignancies could be treated concurrently or consecutively with tumor antigen specific TCR and CA constructs (e.g. TyrTCR and CSPG4-CAR). These experiments evaluate the applicability of the in vivo T cell genetic engineering for these types of malignancies using intradermal B16/A2 melanoma and B cell lymphoma.

Four cohorts of HLA-A2 and wild type C57BL6 mice (2 cohorts each) are intradermally inoculated with respective malignant cells. Lesions are inoculated in right and left flanks. When established, lesions on the right site are treated with Tyr-TCR and CD19-CAR under optimized conditions. Treated and untreated lesions are monitored by caliper measurements. Treatment continues until treated (or both) lesions regress. Tumor-free mice are kept up to 100 days from the beginning of the treatment and monitored for the development of secondary lesions. Then, half of the tumor-rejecting animals are euthanized and splenocytes are examined for the presence and the phenotype of the Tyr-TCR+ and CD19-CAR+ T cells in respective cohorts. The remaining half of the animals in each treatment group receive a challenging inoculation of the respective malignant cells. Rejection of the challenge and analysis of splenocytes are indicative of the acquisition of the protective immunologic memory. All animals rejecting secondary tumors are kept for additional 100 days, monitored and then-used for the assessment of the Tyr-TCR+ and CD19-CAR+ memory T cells as described elsewhere herein.

Targeting of Metastatic Melanoma with Different Tumor-Targeting Receptors.

Recombinant TCR T cells showed remarkable success in targeting solid tumors, particularly melanoma in clinical settings (Phan et al., Cancer control: journal of the Moffitt Cancer Center 2013, 20:289-297). To date, several TCR developed against melanocytic cell-specific proteins including tyrosinase, gp-100, and MARTI were successfully tested in clinical settings for the targeting of stage IV melanoma via ACT. With the accumulation of the TCR coding sequences, the described technology may permit concurrent or consequent generation of the recombinant T cells for the targeting of multiple tumor antigens. However, TCR activity is restricted by the HLA molecules and antigen expression both of which could be down-regulated in tumors (Garrido et al., Immunology today 1997, 18:89-95).

To avoid these limitations, in vivo T cell genetic engineering also offers a possibility to concurrently or consequently generate T cells expressing tumor-reactive TCR and CAR. To experimentally test this approach, pulmonary melanoma metastases are inoculated using a mixture of the B16/A2-CSPG4 and B16F0-CSPG4 melanoma cells. Then, mice receive concurrent treatments with Tyr-TCR and CSPG4-CAR constructs into multiple spots. Based on prior data, (Igoucheva et al., Gene therapy 2013, 20:939-948; Davila et al., PloS one 2013, 8:e61338), that the primary efficacy endpoint is anticipated to be the 4-week survival of the treated mice. After 4 treatments (total of 5 weeks from 1st treatment) half of the experimental animals are euthanized for analysis of the pulmonary lesions and the presence of the Tyr-TCR+ and CSPG4-CAR+ T cells. If targeting of the tumors by both constructs is observed, remaining half of the experimental mice are monitored up to 100 days and receive challenging intradermal inoculation of both HLA-A2-positive and negative, CSPG4+ cells.

Inoculation sites are monitored for the progression of the lesions and pigmentation changes in B16/A2-CSPG4 inoculation sites. Antigen-specific immunologic memory T cells are examined in mice rejecting secondary challenge by immuno-phenotyping of the recombinant TCR+/CAR+ splenic T cells and adoptive transfer of these cells into mice bearing respective tumor lesions as previously described (Novak et al., Molecular cancer therapeutics 2007, 6:1755-1764). Further improvement of the treatment if necessary, could be achieved by increasing the frequency of treatments (twice a week), which could be tested in additional experiments.

### Targeting of Systemic B Cell Lymphoma Via Intradermal Engineering of the CD19-CAR T Cells.

In vivo T cell genetic engineering of the CD19-CAR T cells has the potential to provide benefits that are compatible with ACT in B cell lymphoma bearing mice and improve their survival as it was demonstrated previously (Davila et al., PloS one 2013, 8:e61338). Experiments are conducted where 2 cohorts of the control and experimental wild type animals are i.v. injected with B cell lymphoma and treated with CD19-CAR construct as described elsewhere herein under optimized conditions. Blood from control and experimental animals are collected by the retro-orbital bleeding every week for 10 weeks. Bone marrow function is evaluated by the white cell count, hemoglobin, and platelets measurements and analysis. Persistence of B cell lymphoma, normal B cells and CD19-CAR T cells in circulation is analyzed as described elsewhere herein. All tumor-rejecting mice are monitored for at least 100 days after first treatment for signs indicative of any complication. Then, tumor-rejecting animals are challenged with intradermal inoculation of the B cell lymphoma and the site of injection is monitored for tumor development. Rejection of the tumor challenge and the presence of the CD19-CAR+ T cells with central memory phenotype (CD44+, CD62L+, CCR7+) in the spleens or in BM, as demonstrated in ACT animal studies (Davila et al., PloS one 2013, 8:e61338), demonstrate acquisition of the protective immunologic memory.

Availability of the experimental data obtained in animal studies on melanoma and B cell lymphoma using recombinant TCR and CAR-modified T cells for the ACT (Frankel et al., J Immunol 2010, 184:5988-5998; Davila et al., PloS one 2013, 8:e61338) allows for the comparison of the efficacy of the proposed technology with currently established methods.

The experiments described herein provide pre-clinical data on the utility of the described technology for the treatment of localized and metastatic/systemic melanoma and B cell lymphoma. It is anticipated that intralesional treatment leads to the engineering of the tumor-reactive T cells which are able to egress from the treated tumor and target distal lesions as well as generate protective peripheral and central memory. Strategies to improve systemic immunity are developed and adopted for the treatment. Further, experiments are conducted to examine concurrent treatment with melanoma-specific TCR and CAR. This allows for the comparison of CAR and TCR capacity to target metastatic HLA-A2-positive and negative lesions. If at 5 week time point a substantial tumor regression is not observed but recombinant TCR and CAR T cell are detected in sentinel mice, the remaining half of the experimental mice will receive additional 4 treatments prior to the follow-up analysis. If concurrent treatment is successful, additional experiments are set up to test consequent TCR and CAR treatments. The present experiments generate sufficient evaluative pre-clinical data of the targeting of the systemic B cell lymphoma.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method for treating cancer in a subject in need thereof, comprising:
(a) administering a cytokine composition at a treatment site of the subject thereby recruiting at least one T cell to the treatment site; and
(b) administering an antigen receptor composition to the subject to genetically modify the recruited at least one T cell to express an antigen receptor.

2. The method of claim 1, wherein
(a) the cytokine composition comprises a recombinant cytokine or nucleic acid molecule encoding a cytokine;
(b) the antigen receptor composition comprises an isolated nucleic acid molecule comprising a nucleic acid sequence encoding an antigen receptor; or
(c) a combination thereof.

3. The method of claim 2, wherein
(a) the cytokine is selected from the group consisting of CCL2, CCL3, CCL4, CCL5, macrophage inflammatory proteins (MIP-1α), CXCL9, CXCL10, CXCL12, CXCL16, CCL17, CCL19, CCL20, CCL21, CCL22, and CCL27;
(b) the antigen receptor is a T cell receptor (TCR) or chimeric antigen receptor (CAR); or
(c) a combination thereof.

4-5. (canceled)

6. The method of claim 1, wherein the antigen receptor composition is administered at the treatment site.

7. The method of claim 2, wherein the method further comprises administering an integration composition to the subject, wherein the integration composition induces the integration of the nucleic acid sequence encoding the antigen receptor into the DNA of the recruited at least one T cell.

8. The method of claim 7, wherein the integration composition comprises an integrase, nucleic acid molecule encoding an integrase, recombinase, or nucleic acid molecule encoding a recombinase.

9. The method of claim 1, wherein administration of the cytokine composition recruits a diverse population of T cells or a pre-defined subset of T cells.

10. (canceled)

11. The method of claim 1, wherein the treatment site is the skin or a tumor of the subject.

12. The method of claim 2, wherein
(a) the nucleic acid molecule encoding a cytokine is administered using electroporation;
(b) the nucleic acid molecule encoding an antigen receptor is administered using electroporation; or
(c) a combination thereof.

13. (canceled)

14. The method of claim 8, wherein the nucleic acid molecule encoding an integrase or the nucleic acid molecule encoding a recombinase is administered using electroporation.

15. A method for generating a tumor-specific T cell in a subject, comprising:
(a) administering a cytokine composition at a treatment site of the subject thereby recruiting at least one T cell to the treatment site; and
(b) administering an antigen receptor composition to the subject to genetically modify the recruited at least one T cell to express an antigen receptor that binds to a tumor-specific antigen.

16. The method of claim 15, wherein
(a) the cytokine composition comprises a recombinant cytokine or nucleic acid molecule encoding a cytokine;
(b) the antigen receptor composition comprises an isolated nucleic acid molecule comprising a nucleic acid sequence encoding an antigen receptor; or
(c) a combination thereof.

17. The method of claim 16, wherein
(a) the cytokine is selected from the group consisting of CCL2, CCL3, CCL4, CCL5, macrophage inflammatory proteins (MIP-1α), CXCL9, CXCL10, CXCL12, CXCL16, CCL17, CCL19, CCL20, CCL21, CCL22, and CCL27;
(b) the antigen receptor is a T cell receptor (TCR) or chimeric antigen receptor (CAR); or
(c) a combination thereof.

18-19. (canceled)

20. The method of claim 15, wherein the antigen receptor composition is administered at the treatment site.

21. The method of claim 16, wherein the method further comprises administering an integration composition to the subject, wherein the integration composition induces the integration of the nucleic acid sequence encoding the antigen receptor into the DNA of the recruited at least one T cell.

22. The method of claim 21, wherein the integration composition comprises an integrase, nucleic acid molecule encoding an integrase, recombinase, or nucleic acid molecule encoding a recombinase.

23. The method of claim 15, wherein administration of the cytokine composition recruits a diverse population of T cells or a pre-defined subset of T cells.

24. (canceled)

25. The method of claim 15, wherein the treatment site is the skin or a tumor of the subject.

26. The method of claim 16, wherein
(a) the nucleic acid molecule encoding a cytokine is administered using electroporation;
(b) the nucleic acid molecule encoding an antigen receptor is administered using electroporation; or
(c) a combination thereof.

27. (canceled)

28. The method of claim 22, wherein the nucleic acid molecule encoding an integrase or the nucleic acid molecule encoding a recombinase is administered using electroporation.
